(19) United States
(12) Patent Application Publication
(10) Pub. No.: US 2013/0210049 A1
(43) Pub. Date: Aug. 15, 2013

(54) Polymeric Support With Nanofeatures for Cell Culture

(71) Applicants: Melinda LARSEN, Albany, NY (US); Riffard JEAN-GILLES, Albany, NY (US); Sharon SEQUEIRA, Menands, NY (US); David SOSCIA, Slingerlands, NY (US); James CASTRACANE, Albany, NY (US)

(72) Inventors: Melinda LARSEN, Albany, NY (US); Riffard JEAN-GILLES, Albany, NY (US); Sharon SEQUEIRA, Menands, NY (US); David SOSCIA, Slingerlands, NY (US); James CASTRACANE, Albany, NY (US)

(21) Appl. No.: 13/838,882

(22) Filed: Mar. 15, 2013

Related U.S. Application Data
(63) Continuation-in-part of application No. PCT/US2011/052011, filed on Sep. 16, 2011.
(60) Provisional application No. 61/383,452, filed on Sep. 16, 2010.

(51) Int. Cl. C12N 5/071

(52) U.S. Cl. CPC C12N 5/0633
USPC 435/22; 435/377; 435/29; 435/289.1

(57) ABSTRACT

The invention provides a cell support device comprising a nanofiber structure disposed on a concave surface of a substrate. and the curvature of the substrate in combination with the nanotopography provided by the nanofiber support provides the necessary environmental cues that promote organization, growth, differentiation and morphogenesis of secretory epithelial cells, such as salivary gland epithelial cells. The nanotopography of the device is influenced by features of the nanofiber structure including nanofiber diameter, pore size, biochemical modification and curvature.

### CROSS-REFERENCE TO RELATED APPLICATIONS

This application is a continuation-in-part of PCT/US2011/052011 filed on Sep. 16, 2011 and published in English as WO 2012/037505 on May 31, 2012, which claims priority to U.S. provisional application Ser. No. 61/383,452 filed Sep. 16, 2010, Ser. No. 61/392,670 filed Oct. 13, 2010, and Ser. No. 61/420,035 filed Dec. 6, 2010; the contents of each are hereby incorporated by reference in their entirety into the present application.

### STATEMENT OF RIGHTS UNDER FEDERALLY-SPONSORED RESEARCH

This invention was made with government support under grant nos. R21DE01919701 and 1R01DE022467 awarded by the NIDCR of the National Institutes of Health. The government has certain rights in the invention.

### FIELD OF THE INVENTION

The present invention relates to supports for cellular attachment, growth, differentiation and morphogenesis. The invention also relates to methods of making and using these supports for tissue engineering and high throughput screening. In particular, the present invention relates to a structure to support the growth, differentiation and morphogenesis of secretory epithelial cells, such as salivary gland epithelial cells.

### BACKGROUND OF THE INVENTION

The need for technologies that will maintain, improve or even restore the function of diseased organs is growing. One example of such a disease is xerostomia, a condition that results from salivary gland hypofunction (insufficient saliva production) and which is a significant clinical problem in the United States. Xerostomia causes a decreased quality of life as the result of multiple symptoms, including increased dental caries, oropharyngeal infections, difficulties with swallowing (dysphagia) and digestion (mucositis), loss of taste, and pain. There are three primary causes of xerostomia: 1) Sjogren's Syndrome, an autoimmune disease affecting 1-4 million Americans, 2) radiation therapy (60,000 head and neck cancer patients diagnosed annually in the United States) [2], and 3) combinatorial side effects of medications. Patients suffering from these conditions experience a decreased quality of life resulting from multiple symptoms, yet the current treatment methods are inadequate [1].

There is currently a significant clinical need for artificial salivary glands to relieve symptoms in patients suffering from xerostomia and therefore, considerable interest in creating an artificial salivary gland. An engineered functional salivary gland could provide relief for these patients.

One of the most significant challenges currently faced by tissue engineering is the creation of complex 3D structures that functionally replicate native tissues. Maintenance of salivary acinar cell differentiation and function in vitro is critical to the successful engineering of such constructs; however, this breakthrough has not yet been achieved. This is primarily due to the current lack of basic scientific knowledge regarding the specific extracellular signals regulating acinar cell differentiation, which remains a substantial limitation in the ability to engineer a functional artificial salivary gland. Previous work has not produced an appropriate environment that successfully mimics the native extracellular matrix to support growth and differentiation of salivary acinar cells [3, 4].

### SUMMARY OF THE INVENTION

The present invention represents the outcome of an innovative interdisciplinary strategy combining the synthesis of nanofibers with micro- and nano-scale patterning to create a cell support structure that promotes secretory epithelia cell differentiation and tissue development ex vivo. By independently modulating nanotopography, micropatterning, and chemical signaling, the contributions of each factor is optimized to engineer constructs that support acinar cell differentiation and function.

The present invention provides novel and improved compositions and methods for the growth, differentiation and morphogenesis of secretory epithelial cells into functional units by providing a cell support structure that approximates the mechanical and chemical properties of the natural structural environment needed to facilitate development toward acinar formation.

In one aspect, therefore, the invention relates to a cell support comprising a substrate or array with one or more concave surfaces and comprising a nanofiber structure disposed upon a concave surface of a substrate, for example, a substrate that has been micropatterned with one or more depressions or craters to form one or more concave surfaces. The curvature of the concave surface will be defined by the aspect ratio applicable to the depressions or craters, that is, the ratio of the depth of the crater to the radius of the crater. The craters of the substrate have a radius in the range of about 10 μm to about 100 μm; in one embodiment, the craters have a radius in the range of about 10 μm to about 50 μm; in one embodiment, the craters have a radius in the range of about 20 μm to about 30 μm. Depth of the craters is in the range of about 20 μm to about 40 μm. The concave surface(s) of the substrate or array may have a curvature with an aspect ratio in the range of about 0.5 to about 1; or in the range of about 0.6 to about 1; or in the range of about 0.8 to about 1. In degrees, the concave surface(s) of the substrate or array may have a curvature of between about 10 to about 30 degrees. In some embodiments, the curvature is between about 15 and 28 degrees; in yet other embodiments, the curvature is between about 25 and 30 degrees.

In a related aspect, the invention relates to a cell support comprising a nanofiber structure that has been created by electrospinning polymers selected from synthetic polymers, natural polymers, protein engineered biopolymers, biodegradable or non-biodegradable polymers or combinations thereof on top of the concave structure in a surface of the substrate. Optionally, the polymer used to generate the nanofibers is derivatized with active groups (e.g. carboxylic, hydroxyl or amino groups or the like) so that the chemical and/or mechanical properties of the polymer may be modified before or after formation of the nanofiber structure, for example, to facilitate the incorporation of biological molecules into the nanofiber structure. Functionalization of the nanofiber structure can be achieved by the addition of a component of the extracellular matrix, for example, fibronectin, collagen, elastin, laminin, chitosan, perlacan and the like or a combination thereof.

In yet another related aspect, the invention relates to a cell support comprising a nanofiber structure comprising a polymer selected from the group consisting of silk, laminin, poly(∈-caprolactone) (PCL), poly(∈-caprolactone-co-ethyl ethylene phosphate) (PCLEEP), poly(lactic acid) (PLA), poly(lactic-co-glycolic acid) (PLGA), poly(lactic acid-co-∈-caprolactone) (PLACL), and polydioxanone (PDO), poly acrylamide (PAAM), poly acrylic acid (PAA), poly acrylonitrile (PAN), poly amide (Nylon) (PA, PA-4,6, PA-6,6), poly aniline (PANI), poly benzimidazole (PBI), poly bis(2,2,2-trifluoroethoxy) phosphazene, poly butadiene (PB), poly carbonate (PC), poly ether amide (PEA), poly ether imide (PEI), poly ether sulfone (PES), poly ethylene (PE), poly ethylene-co-vinyl acetate (PEVA), poly ethylene glycol (PEG), poly ethylene oxide (PEO), poly ethylene terephthalate (PET), poly ferrocenyldimethylsilane (PFDMS), poly 2-hydroxyethyl methacrylate (HEMA), poly 4-methyl-1-pentene (TpX), poly methyl methacrylate (pMMA), poly p-phenylene terephthalamide (PPTA), poly propylene (PP), poly pyrrole (PPY), poly styrene (PS), polybisphenol-A sulfone (PSF), poly sulfonated styrene (PSS), Styrene-butadiene-styrene triblock copolymer (SBS), poly urethane (PU), poly tetrafluoro ethylene (PTFE), poly vinyl alcohol (PVA), poly vinyl carbazole, poly vinyl chloride (PVC), poly vinyl phenol (PVP), poly vinyl pyrrolidone (PVP), and poly vinylidene difluoride (PVDF).

In another aspect, the invention relates to a method of making a cell support of the invention comprising providing a substrate having a concave surface or more than one concave surface and using a process, such as electrospinning, to dispose a nanofiber structure over the concave surface of the substrate.

In yet another aspect, the invention relates to a method for promoting differentiation and morphogenesis of secretory epithelial cells ex vivo, the method comprising seeding secretory epithelial cells on the nanofiber structure of the cell support of the invention; and incubating said cell support and cells under conditions sufficient for proliferation, differentiation and morphogenesis of said cells to occur.

In a related aspect, the invention relates to a device and method for screening compounds that modulate secretory epithelial cell development. The device comprises a substrate having a concave surface, a nanofiber structure disposed over the concave surface of the substrate and secretory epithelial cells seeded on the nanofiber structure of the device. The method comprises providing a device comprising a substrate having a concave surface and a nanofiber structure disposed on the concave surface of the substrate, seeding secretory epithelial cells on the nanofiber structure of said cell support; incubating said cell support and cells under conditions otherwise sufficient for proliferation, differentiation and morphogenesis to occur in the presence and absence of the compound to be tested; monitoring development of cells in the presence of the compound and monitoring development of cells in the absence of the compound and comparing the development of cells grown in the presence of the compound with the development of cells grown in the absence of the compound to determine the effect of the test compounds on development, proliferation, and differentiation and acquisition/maintenance of cell polarity.

In one aspect, the invention relates to a method of screening a test compound that modulates development of secretory epithelial cells by evaluating parameters of development such as cell morphology of the cells seeded on the nanofiber structure; expression of differentiation markers such as ZO-1; production of organ product such as proteins found in saliva or pancreatic fluid or any combination of these parameters.

In a related aspect, the invention is directed to an in vitro method for determining the effect of a test compound on the development of salivary gland cells.

In yet another related aspect, the invention is directed to an in vitro method for determining the effect of a test compound on the development of exocrine pancreas cells.

### BRIEF DESCRIPTION OF THE DRAWINGS

FIG. 1 contains a schematic showing the structure of an exocrine gland (from Gartner, L. P. and J. L. Hiatt, 2005. Color Atlas of Histology. 5th Edition. Lippincott, Wiliams and Wilkins.

FIG. 2 is a schematic comparing the effects of growing secretory epithelial cells on a flat versus a curved support.

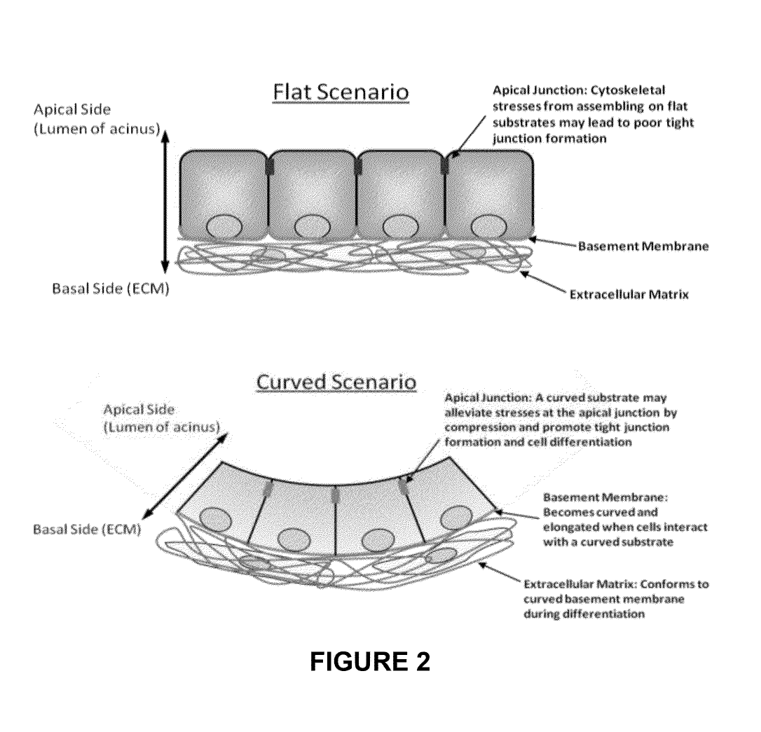

FIGS. 3A-C show the difference in cell-matrix adhesions that occur in salivary epithelial cells growing on different supports. (A) are confocal images of cells on different surfaces stained for nuclei (DAPI in blue), Fak, actin and Talin; (B) Western blots for p-Fak and Talin showing reduced Fak phosphorylation and talin expression on nanofibers vs. microfibers or flat surfaces; and (C) densitometric analysis of blots in (B).

FIGS. 4A-B show that PLGA fibers promote cell clustering. (A) Confocal images of SIMS submandibular gland cell nuclei (DAPI staining) as an indicator of cell spreading; and (B) quantitative analysis of cell clustering using cell graph metrics shows an increasing trend of cell clustering on fiber surfaces as compared to flat glass and PLGA film surfaces.

FIG. 5 shows that focal adhesion proteins are expressed at low levels and are diffusely localized in mature adult salivary glands.

FIGS. 6A-B show an SEM of (A) a substrate with multiple craters each about 70 μm in diameter on which nanofibers have been spun and (B) a close up view of one of the craters showing that the nanofiber structure conforms to the shape of the crater.

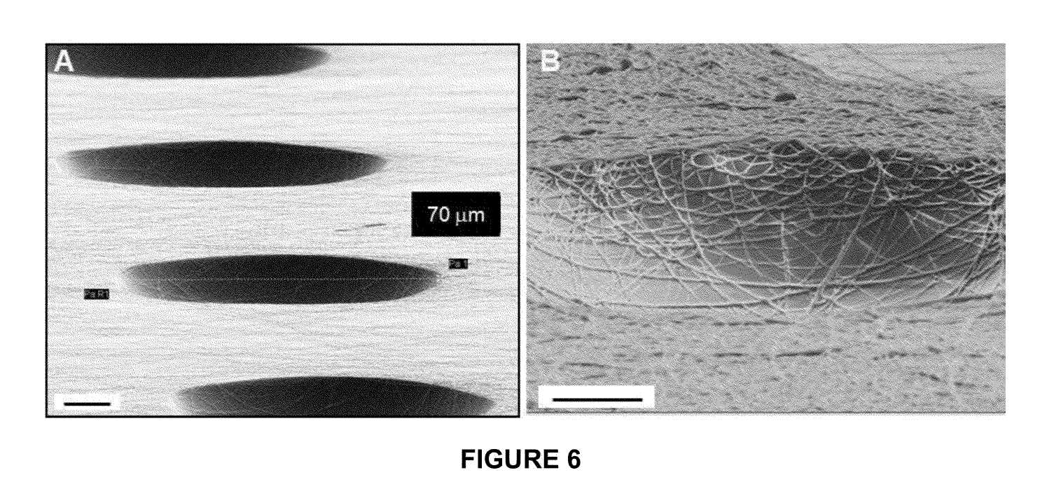

FIGS. 7A-E are SEM of nanofibrous crater arrays. (A-B) After thermal reflow, photoresist mound arrays are highly ordered and uniform. (C) Plot shows a decrease in degree of curvature as feature radius increases (“Crater radius” refers to the radius of the circle used in the photolithography mask and is independent from the height of the mounds or the depth of the craters). (D) After transferring the arrays to PDMS, craters are formed which exhibit the negative shape and same order as the mounds. (E) Once PLGA nanofibers have been deposited onto craters and incubated in PBS for 3 days and cell media for 1 day, fibers conform to the crater curvature and supports are ready for cell seeding. As a result of fiber stretching during conformation, pore size of fibers deposited on the flat areas (Ei) are smaller than those within the crater (Eii).

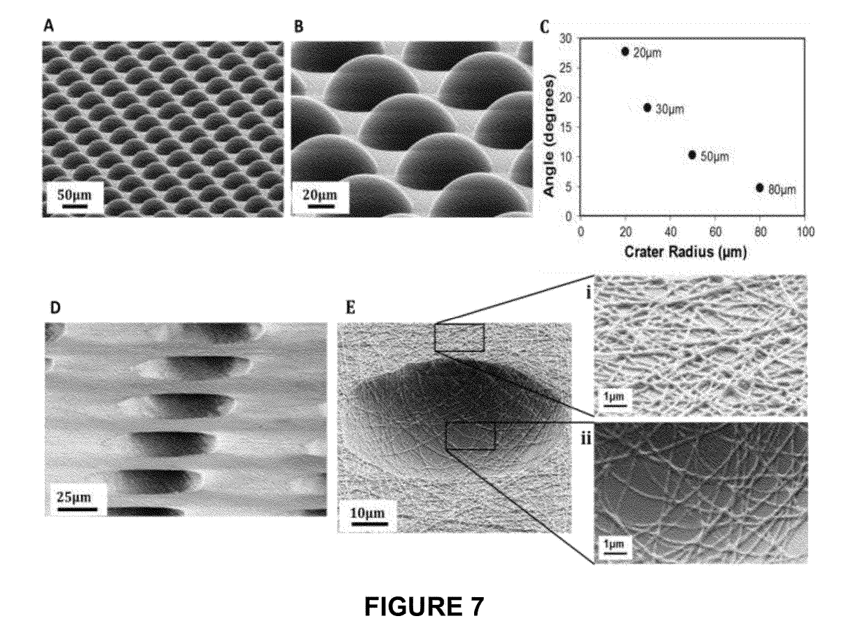

FIG. 8 shows a plot of aspect ratio (another measure of curvature) to radius of the crater or depression.

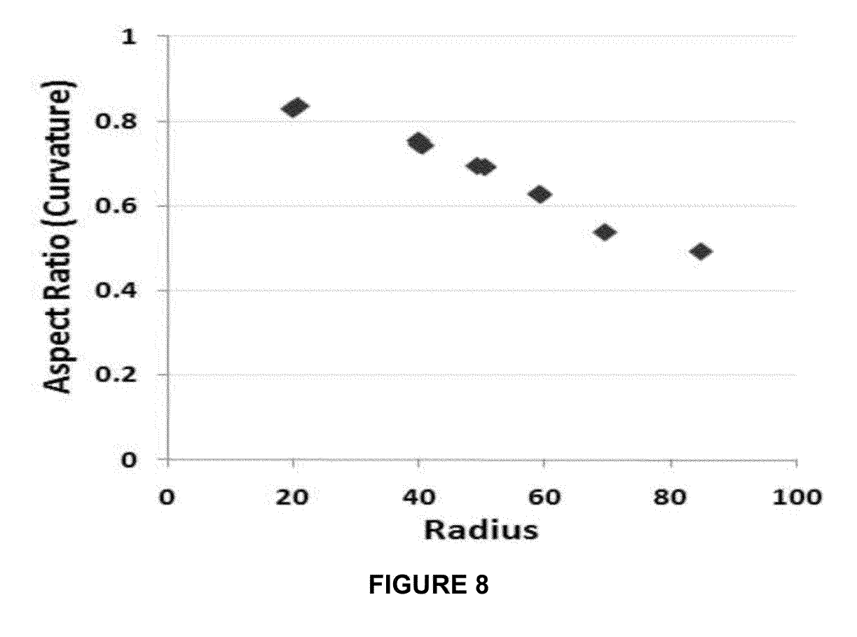

FIG. 9A-D show the (A) viability of cells grown on a glass substrate, a PLGA film and PLGA nanofibers; (B) the total number of cells on a glass substrate, a PLGA film and PLGA nanofibers at 24 and 48 hours; (C-D) cell organization (or lack thereof) on the three surfaces.

FIG. 10A-G show patterns of SIMS cell growth on nanofibrous craters of different sizes. The SIMS mouse submandibular cell line conforms to the shape of 30 μm radius nanofibrous craters after 96 hr of growth. SEM images show cell conformity from (A) top-down and (B) angled views. (C) A tilted view of a fluorescence confocal Z-stack 3-D projection with SIMS stained for f-actin (green, phalloidin), and nuclei (blue, DAPI), conforming to a crater lined with nanofibers (red). The Z-plane is denoted by the arrow. The arrangement of nuclei on (D) 30, (E) 50, (F) 80 μm radius craters, and (G) flat nanofibers. Nuclei preferentially localize within the craters, most obviously on 30 μm radius craters. On 50 and 80 μm radius craters, some regions exhibited cell overlapping (red arrows), whereas on 30 μm radius craters and flat nanofibers, cells grew largely as one monolayer over the entirety of the samples.

FIG. 11 are micrographs of cells grown on cell support with 30 μm radius craters showing smaller nuclear size in cells growing within the crater (blue, DAPI) indicating less cell spreading; and higher expression of a marker for tight junctions.

FIG. 12 shows diffuse distribution of ZO-1, a marker for tight junctions, in cells grown on a flat nanofiber structure.

FIG. 13 shows the localization of ZO-1 in cells grown on a concave surface of a crater with a radius of 80 μm.

FIG. 14 shows increasingly localized ZO-1 at the apical portion of cells grown on a concave surface of a crater with a radius of 30 μm.

FIG. 15 is a composite of FIGS. 11 through 13 comparing the distribution of ZO-1 in cells grown on flat and concave surfaces.

FIG. 16 shows the Z-height of cells grown on flat nanofiber structures and nanofiber structures disposed on craters with a radii of 30 μm, 50 μm and 80 μm.

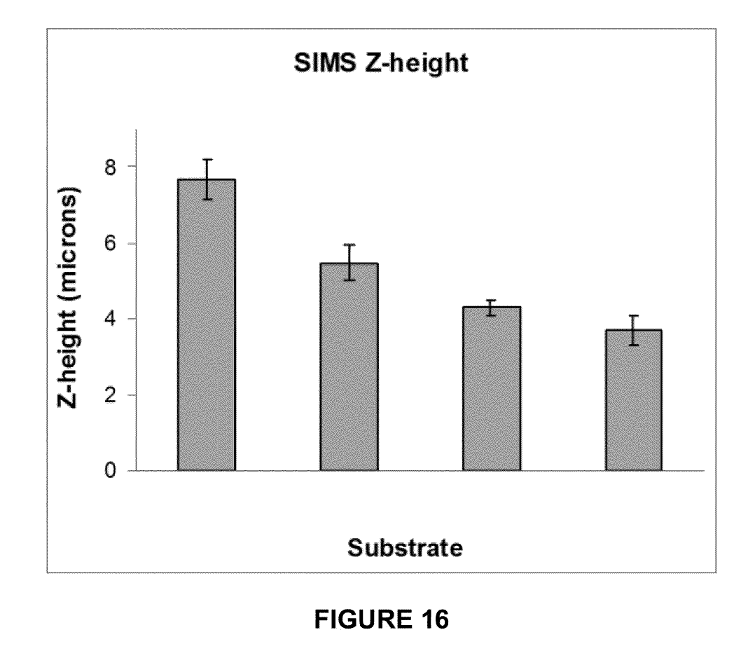

FIG. 17A-D Confocal top-down and cross-sectional views illustrating differences in monolayer height of SIMS cell monolayer on a (A) 30 μm radius crater (B) flat nanofibers, and in (C) 3-D Matrigel respectively. Measurements taken in craters were conducted at the center (bottom) of the craters. Cells on fibers are stained for F-actin (phalloidin, green) and nuclei (DAPI, blue). Nanofibers are stained red. Cells in Matrigel were stained for F-actin (green, phalloidin), and nuclei (DAPI, blue). (D) Measured heights of SIMS cell monolayers on craters of various sizes, standard nanofibers, and Matrigel. Thin Matrigel is a layer of Matrigel deposited such that it is too thin for cells to penetrate into and grow in full 3-D, whereas 3-D Matrigel is deep enough for cells to grow within the gel. *** indicates data point is significantly different from others (ANOVA, p<0.001).

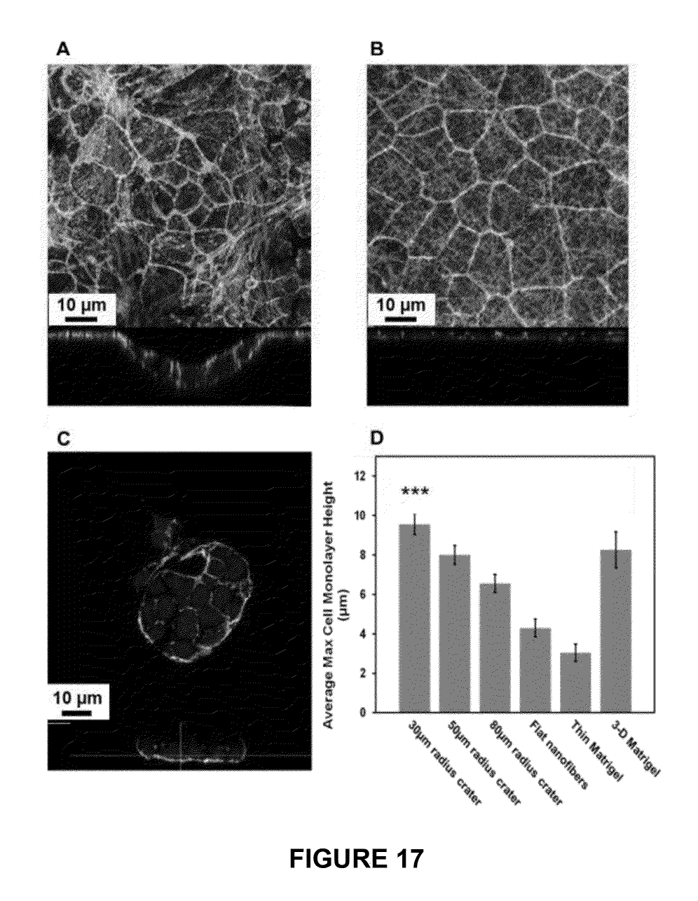

FIG. 18A-C (A) Confocal top-down images of nuclei (DAPI, blue) and expression of tight junction protein occludin (green) in SIMS cells grown on nanofibrous substrates of varying curvature. The protein is expressed and localized to cell membranes on all substrate types. (B) Cross-sectional views of confocal z-stacks stained for nuclei (DAPI, blue), occludin (green), and fibers (red) show that occludin is more apically-localized (top of the cells) in SIMS on 30 μm radius craters as compared to those grown on flat nanofibers, where the protein fluorescence extends from the apical to the basal side of the cells. This trend can be further visualized in (C), where profiles of occludin fluorescent intensity have been plotted for lines drawn though the apical (blue) and basal (red) side of SIMS cell monolayers on 30 μm radius craters and flat nanofibers, respectively. Cells on the crater show markedly larger peaks for a line drawn though the apical region of the cell monolayer as compared to the basal profile, whereas cells on flat nanofibers have similarly-sized peaks for both apical and basal regions.

FIG. 19A-C (A) Confocal top-down images of nuclei (DAPI, blue) and expression of tight junction protein ZO-1 (green) in SIMS cells grown on nanofibrous substrates of varying curvature. The protein is expresses and localized to cell membranes on all substrate types. (B) Cross-sectional views of confocal z-stacks stained for nuclei (DAPI, blue), ZO-1 (green), and fibers (red) show that ZO-1 is more apically-localized (top of the cells) in SIMS on 30 μm radius craters as compared to those grown on flat nanofibers, where the protein fluorescence extends from the apical to the basal side of the cells. This trend can be further visualized in (C), where profiles of ZO-1 fluorescent intensity have been plotted for lines drawn though the apical (blue) and basal (red) side of SIMS cell monolayers on 30 μm radius craters and flat nanofibers, respectively. Cells on the crater show markedly larger peaks for a line drawn though the apical region of the cell monolayer as compared to the basal profile, whereas cells on flat nanofibers have similarly-sized peaks for both apical and basal regions.

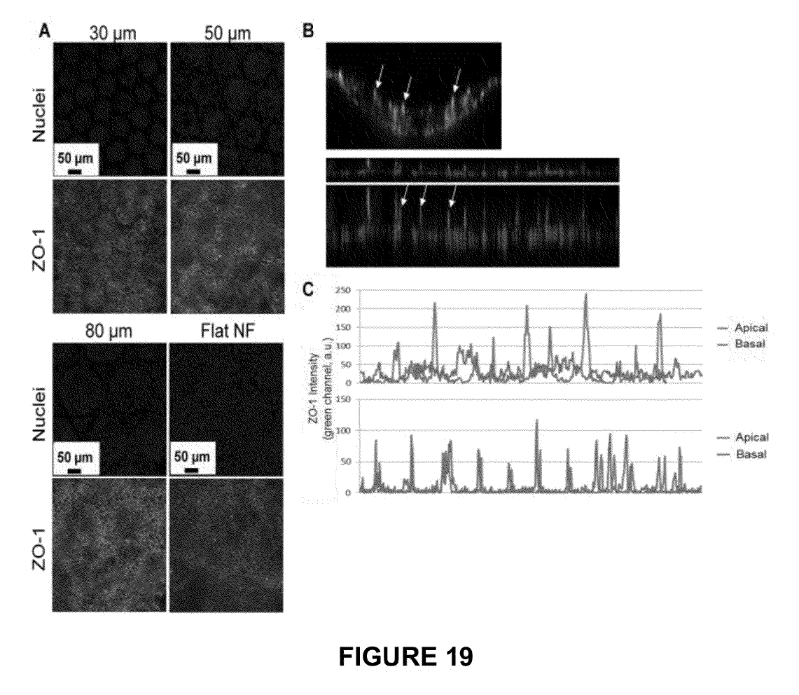

FIG. 20 Expression and localization of adherens junction protein E-cadherin on SIMS cells grown on 30 μm, 50 μm, 80 μm radius craters and flat nanofibers, respectively. The upper images show the arrangement of the cells through staining of the nuclei, and the lower images show E-cadherin staining of cells corresponding to the arrangement of cells in the top row.

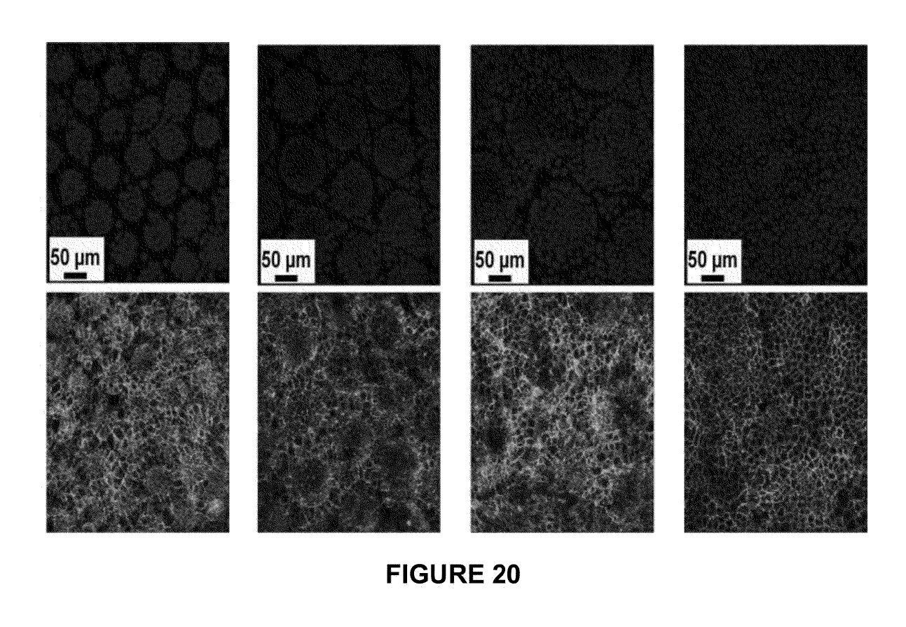

FIG. 21A-H shows Par-C10 cell organization on nanofibrous substrates of varying curvature. The arrangement of nuclei in (A) 30 μm, (B) 50 μm, (C) 80 μm radius craters, and (D) flat nanofibers reveals that Par-C10 cells are more spread than SIMS, and nuclei do not preferentially localize within craters. Confocal top-down and orthogonal views showing height of Par-C10 cell monolayer on (E) 30 μm radius crater (F) flat nanofibers, and (G) 3-D Matrigel respectively. The cross-sectional views show that cell monolayer layer heights for both nanofibrous substrates are distinctly lower than cells grown 3-D Matrigel. Cells are stained for F-actin (phalloidin, green) and nuclei (DAPI, blue). Nanofibers are stained red. When Par-C10 heights were quantified (D), cells grown on 30 μm radius craters had significantly higher monolayer heights than cells on substrates of lesser curvature, and those growing in 3-D Matrigel exhibited markedly higher cell layer heights than any of the other samples (ANOVA *p<0.05, **p<0.01, ***p<0.001).

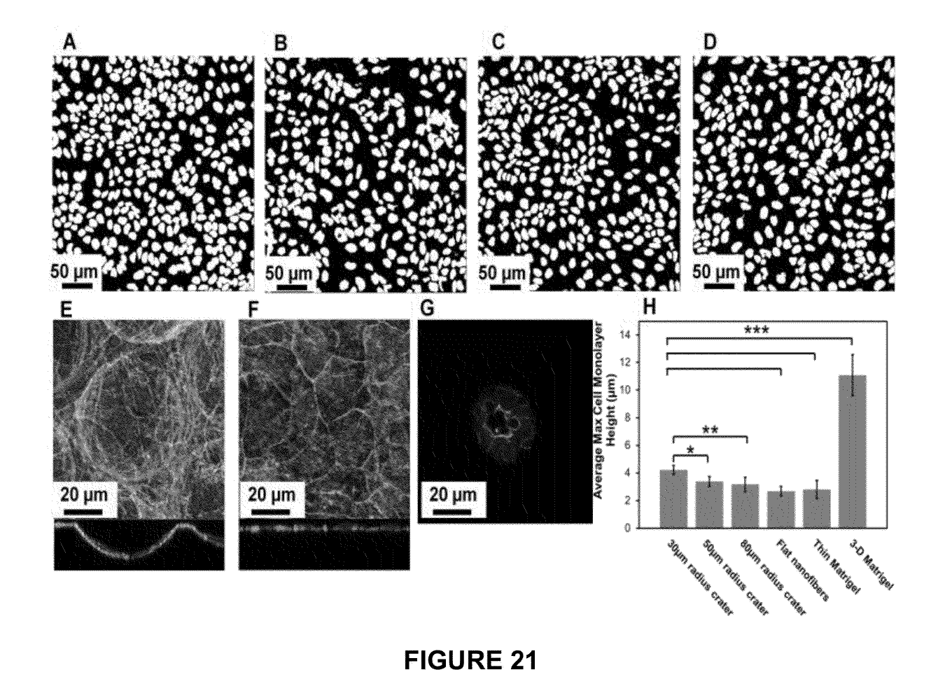

FIG. 22 shows that ParC10 cells show increased height on smaller radius craters.

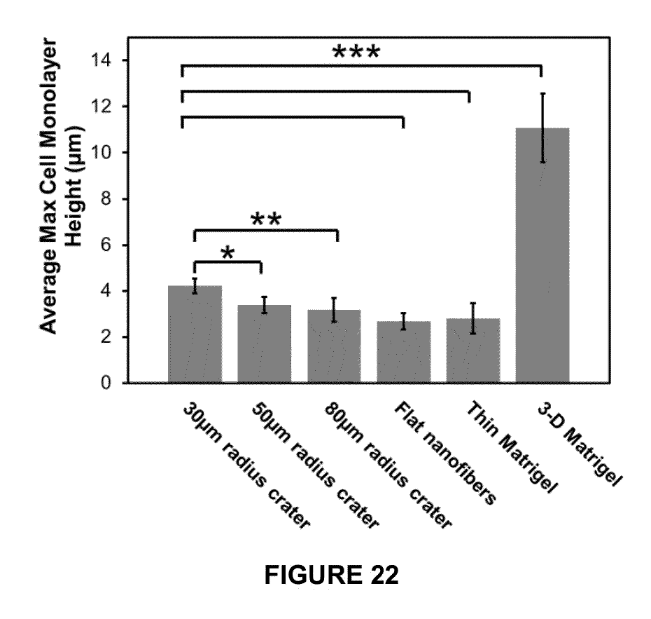

FIG. 23 shows that the water channel Aquaporin-5 (Aqp-5) is expressed at higher levels in ParC10 cells grown on the 30 μm craters than on other scaffolds. A representative Western blot and corresponding normalized band intensity plot showing that expression of Aqp-5 increases when cells are grown on 30 μm craters as compared to substrates of lesser curvature. Protein in the Matrigel itself may have affected total measured protein and contributed to the relatively low intensity seen in the Matrigel band. NIH 3T3 fibroblasts and adult mouse submandibular gland (SMG) tissue were used as negative and positive controls, respectively.

FIG. 24A-D (A) Confocal top-down images of nuclei (DAPI, blue) and expression of tight junction protein occludin (green) in SIMS cells grown on nanofibrous substrates of varying curvature. The protein is expressed and localized to cell membranes on all substrate types. (B) Western blot analysis shows an increase in occludin expression as substrate curvature increases. Normalized band intensities are plotted below blot image. (C) Cross-sectional views of confocal z-stacks stained for nuclei (DAPI, blue), occludin (green), and fibers (red) show that occludin is more apically-localized (top of the cells) in SIMS on 30 μm radius craters as compared to those grown on flat nanofibers, where the protein fluorescence extends from the apical to the basal side of the cells. (D) Profiles of occludin fluorescence intensity for lines drawn though the apical (blue) and basal (red) side of SIMS cell monolayers on 30 μm radius craters and flat nanofibers, respectively. Cells on the crater show markedly larger peaks for a line drawn though the apical region of the cell monolayer as compared to the basal profile, whereas cells on flat nanofibers have similarly-sized peaks for both apical and basal regions.

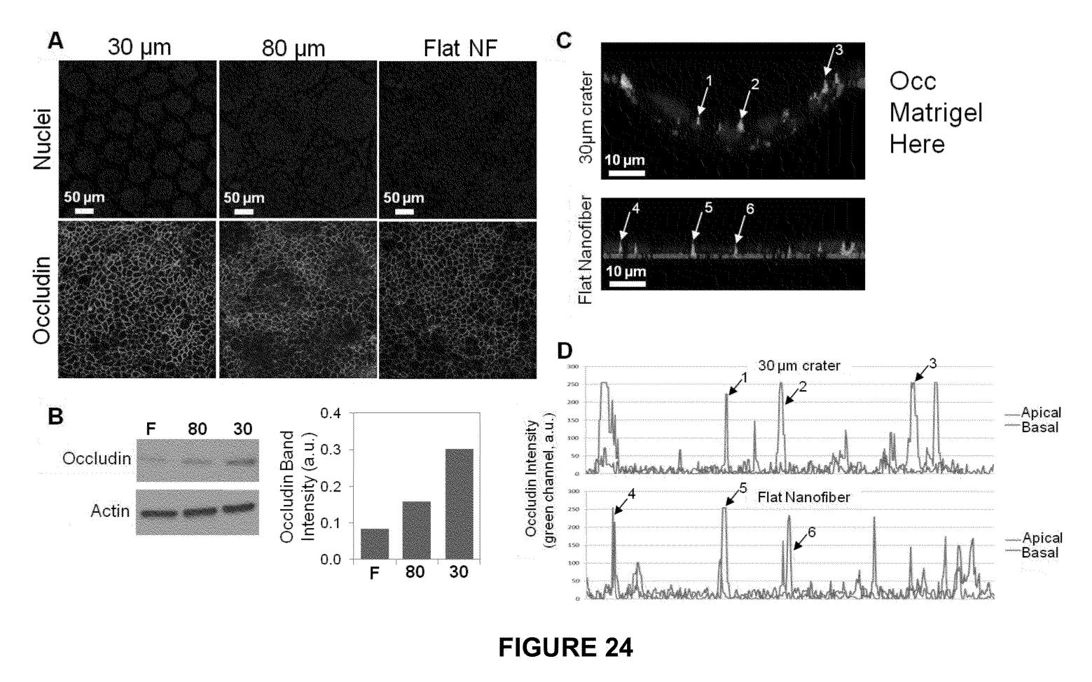

FIG. 25 A-D (A) Confocal top-down images of nuclei (DAPI, blue) and expression of tight junction protein occludin (green) in Par-C10 cells grown on nanofibrous substrates of varying curvature. The protein is expressed and localized to cell membranes on all substrate types. (B) Western blot analysis shows that occludin expression increases as substrate curvature increases, especially on 30 μm radius craters. Normalized band intensities are plotted below blot image. (C) Cross-sectional views of confocal z-stacks stained for nuclei (DAPI, blue), occludin (green), and fibers (red) show that occludin is more apically-localized (top of the cells) in Par-C10s on 30 μm radius craters as compared to those grown on flat nanofibers, where the protein fluorescence extends from the apical to the basal side of the cells. (D) Profiles of occludin fluorescence intensity for lines drawn though the apical (blue) and basal (red) side of Par-C10 cell monolayers on 30 μm radius craters and flat nanofibers, respectively. Cells on the crater show markedly larger peaks for a line drawn though the apical region of the cell monolayer as compared to the basal profile, whereas cells on flat nanofibers have similarly-sized peaks for both apical and basal regions.

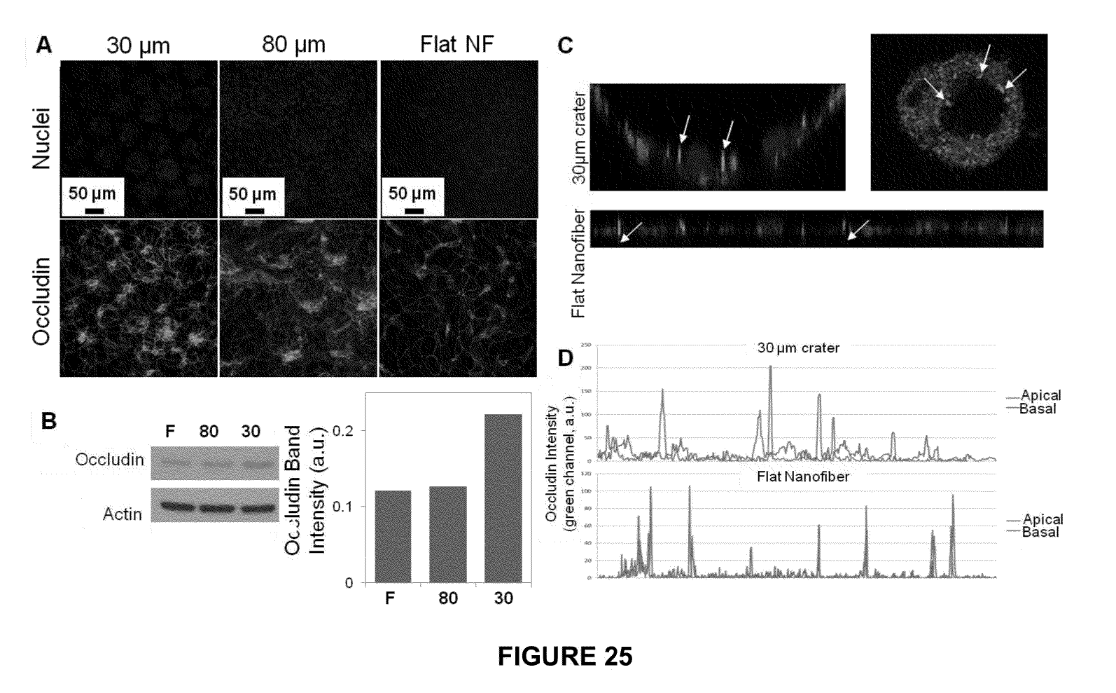

FIG. 26 is a schematic showing how curvature is determined. Curvature in this context is measured in “degree of curvature”, which is the angle between a tangent line drawn at the apex of the curved surface (or mound in our case) and a tangent line drawn from another location on the curved surface defined by travelling a set distance along the first tangent line, then travelling at right angle from this point until the surface of the curve is reached. For our degree of curvature measurements, the distance along the first tangent line was 10 μm for all samples. “Aspect ratio” corresponds loosely to “degree of curvature” in that a feature, or mound in this case, having a higher degree of curvature will have a higher aspect ratio. If the degree of curvature is higher, then the mound curves down toward the surface at a higher angle. This will result in the ratio of its height to its width being larger, or a larger aspect ratio by definition.

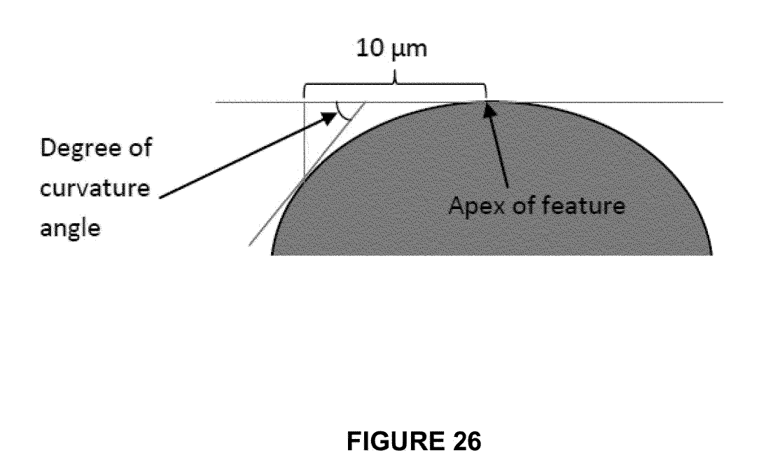

### DETAILED DESCRIPTION OF THE INVENTION

All patents, applications, publications and other references cited herein are hereby incorporated by reference in their entirety into the present application.

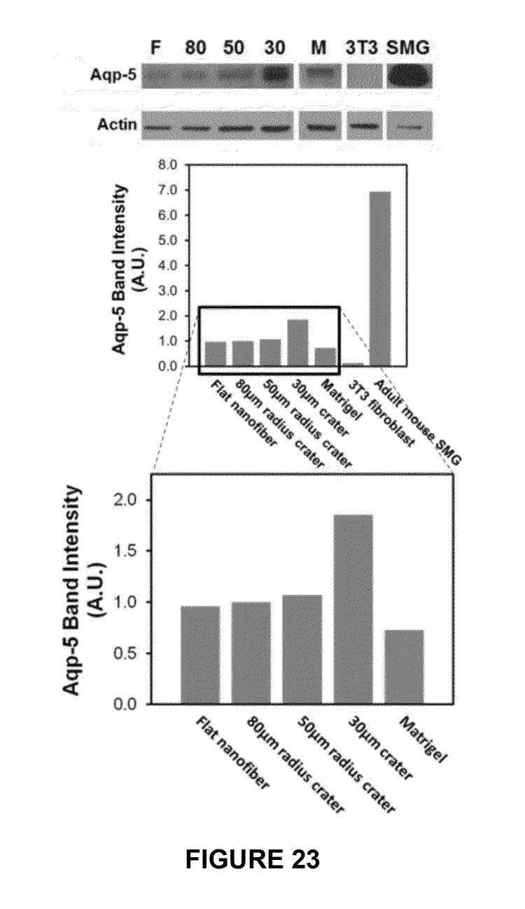

The current invention is an outgrowth of the inventors' observation that nanofibrous structures promote salivary gland cell attachment [5] and cellular organization mimicking the in vivo tissue architecture, demonstrating that substrate nanotopography promotes apical-basal polarity, salivary gland cell differentiation, and saliva secretion. Epithelial secretory cells such as salivary gland cells can be induced to polarize and differentiate on a nanofiber structure having a concave surface with the appropriate curvature. The increase in Aquaporin 5 as shown in FIG. 23) indicates an increase in acinar differentiation on the 30 μm radius craters.

It is the expression of Aqp5 (differentiation marker) in the ParC10 cells that indicates that the salivary gland cells are undergoing acinar cell differentiation moreso than any characteristics of the cellular attachments or other aspects of cellular organization. The engineering of a micropatterned nanofiber structure which can optimize cell attachment, cell organization, and cell differentiation represents a first step in the ultimate development of an artificial exocrine gland.

The present invention represents the first use of a curved nanofiber structure as a substrate for growth of epithelial secretory cells such as salivary gland cells. Polymer nanofibers, such as poly lactic-co-glycolic acid (PLGA) nanofibers, support the attachment, proliferation, and survival of salivary gland epithelial cells. While a PLGA film supported cell attachment and cell growth, the arrangement of PLGA into nanofiber structures promoted the self-organization of cells on this material into cellular aggregates. Additionally, by imparting a curvature to the nanofiber structure, the nanofibers not only are able to facilitate the self-organization of cells into epithelial-like cell clusters or aggregates, but also permit the cells to achieve apical-basal polarity, formation of apically-restricted tight junctions, and expression of proteinacous components of saliva, such as alpha-amylase and salivary androgen binding protein A (SABPA). Although the PLGA nanofibers do not require surface modification to support cell attachment, surface modification and attachment of growth regulatory molecules may be desirable to further modulate acinar cell organization.

Furthermore, this study represents the first use of a systematic design of experiment (DOE) approach towards electrospinning of polymer nanofibers having a predictable and defined diameter for use as cellular scaffolds. This approach is more efficient than evaluating parameters individually because it allows factors to be evaluated in concert, but still allows the effect of each parameter to be individually quantified. In one embodiment of the invention, a transfer function generated for a PLGA-HFIP solution with 1% NaCl was validated, as uniform fibers of predicted diameter were able to be electrospun using parameters suggested by the transfer function. The method can be used to reproducibly generate nanofibers composed of different polymers for generating the cell support of the invention.

### DEFINITIONS

The term “development” refers to cell development, that is, the progression of cells from single cells or populations of single cells through various stages of cell growth, differentiation and morphogenesis that accompany tissue development as it is normally seen in vivo. These include organization of the cells, for example, into clusters, cell differentiation and morphogenesis, including acquisition of apical-basal polarity and columnar morphology. Metrics for the various stages of development of secretory epithelial cells into exocrine tissue are known to those of skill in the art and include, for example, assessment of morphological indicators such as cell shape and polarity, expression of protein markers of differentiation etc.

The term “concave” means curved like a segment of the interior of a circle or hollow sphere surface. A concave surface, therefore, is a surface which has the curvature of a part of a hollow sphere, for example, as a spherical depression in a substrate might. The curvature of a concave surface can be expressed as a “radius of curvature” or as an “aspect ratio.”

The term “aspect ratio,” as used herein, refers to a value obtained by taking the ratio of the depth (or height) of a concave structure, for example a crater or depression in an otherwise planar surface, to its radius. For example, a depression having a radius of 60 μm and a depth of 30 μm will have an aspect ratio of 0.5, whereas, a depression having a smaller radius, for example 30 μm, but the same depth of 30 μm will have an aspect ratio of 1 and greater curvature.

Measurement of curvature is well known in the art. Curvature can be expressed in terms of a “radius of curvature,” that is, the curvature is the reciprocal of the radius of an approximating circle that passes through the points on the curve. Since a line drawn across the widest part of the surface of a depression represents a small section of a circle, the radius of that circle describes the amount of curvature. A surface with a large radius of curvature has little curvature and one with a small radius is highly curved.

“Degree of curvature” is a measure of curvature of a circular arc, for example the arc defined by the curvature of the craters in the substrate. An n-degree curve turns forward direction by n degrees over some agreed-upon distance.

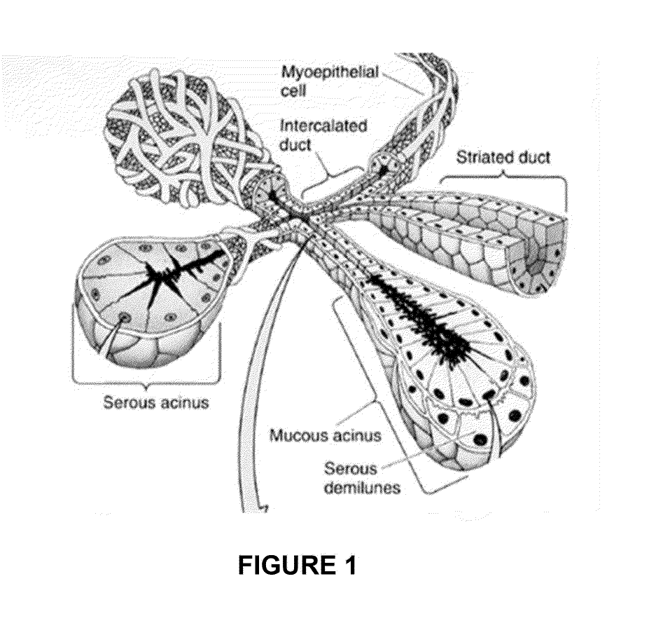

As used herein, the term “epithelial secretory cell” refers to a cell or cells that give rise to a tissue of one of the exocrine systems of the body, for example, exocrine glands such as the salivary glands, sweat glands, sebaceous glands, exocrine pancreas, lacrimal glands, mammary glands and many others. Epithelial secretory cells for use in the present invention, therefore, include, cells derived from an exocrine gland. The epithelial cells of the exocrine glands undergo differentiation and morphogenesis to form a functional structure for example as shown in FIG. 1. The secretory cells in the salivary gland are referred to as acinar cells.

Similarly, “exocrine tissue” is tissue derived from an exocrine gland, that is, an organ of the body that discharges secretions by means of a duct, which opens onto an epithelial surface. Exocrine glands include the salivary, sweat, sebaceous, lacrimal and mammary glands, as well as the exocrine pancreas. Exocrine development is characterized by branching morphogenesis, a process that allows the formation of a branched network of tubes, as exemplified by the excretory ducts of the pancreas and salivary glands. During branching morphogenesis, the epithelial cells organize into polarized monolayers with their apical pole facing the tube lumen. Exocrine glands can be formed by invagination of a sheet of secretory epithelial cells to form a primary bud that then undergoes a developmental process known as branching morphogenesis to form a branched, compound tubular structure. The blind ends of the tube constitute the secretory parts of the gland and may stay tubular or expand to form round sac-like structures called acini or alveoli.

The terms “electrospinning” (commonly referred to as electrostatic spinning) or “electrospun,” as used herein refers to any method where materials are streamed, sprayed, sputtered, dripped, or otherwise turned into a fibrous structure in the presence of an electric field. The electrospun material can be deposited from the direction of a charged container towards a grounded target, or from a grounded container in the direction of a charged target. In particular, the term “electrospinning” means a process in which fibers, usually with diameters in the range of, for example, 10 nm to 100 μm, are formed from a charged solution comprising at least one natural biological material, at least one synthetic polymer material, or a combination thereof by streaming the electrically charged solution through an opening or orifice towards a grounded target.

The term “nanofiber structure” refers to a three-dimensional ultrastructure of interconnected fibers of nanoscale diameter and having pores that may take any form, for example, a mesh, mat, matrix, scaffold or network of nanofibers, that supports cells and, with or without modification, provides the appropriate environmental cues that signal cells to grow, differentiate, organize and develop into tissue characteristic of an exocrine organ or partial organ structure.

The term “biomolecule” as used herein refers to any molecule that is produced by a living organism, including large macromolecules such as proteins, polysaccharides, lipids, and nucleic acids, as well as small molecules such as primary metabolites, secondary metabolites, and natural products.

The novel cell support of the invention is made using methods known to those of skill in the art, including conventional lithographic processing techniques, polymer electrospinning and cell culture methods.

### Preparation of Micro-Patterned Substrate

In one embodiment, the cell support of the invention comprises a micro-patterned substrate having a concave surface upon which a nanofiber structure is disposed. The curvature of the concave surface of the substrate provides the curvature to the overlying nanofiber structure. The substrate comprises any biocompatible material that may be patterned using lithographic or similar techniques known to those of skill in the art.

In one embodiment, a micro-patterned “crater array” is created to form a substrate having multiple concave surfaces upon which to dispose the nanofiber structures. First a silicon master is created. Briefly, a SP-7 positive photoresist is spin-coated onto a silicon wafer that has been cleaned using solutions to remove organic residues, for example, a chemical mixture consisting of sulfuric acid and hydrogen peroxide commonly known as piranha solution, and procedures well known to those in the art. For example, once the wafer has been spin-coated, it is baked at 90° C. for about 5 min., exposed for about 360 seconds, developed and baked again at 150° C. for about 5 min.

Once the cleanroom processing of the silicon master is complete, polydimethylsiloxane (PDMS) is spin-coated onto the wafer at about 600 rpm for about 1 min. The PDMS is cured for an appropriate period of time, for example, for about 2 hours at 60° C., after which the PDMS mold is peeled from the silicon master and cut into individual arrays and prepared to receive nanofibers.

The micro-patterned substrate can be made with depressions (or craters) of various dimensions and curvatures. Generally, a suitable curvature for promoting secretory epithelial cell morphogenesis including attainment of apical-basal polarity, apical localization of markers for tight junctions and columnar morphology is achieved with a depression (or crater) with a radius in the range of about 10-80 μm and aspect ratio between 0.5 and 1; in one embodiment, the radius of the depression is in the range of 20-50 μm; in one embodiment, the aspect ratio is in the range of about 0.8 to 1.

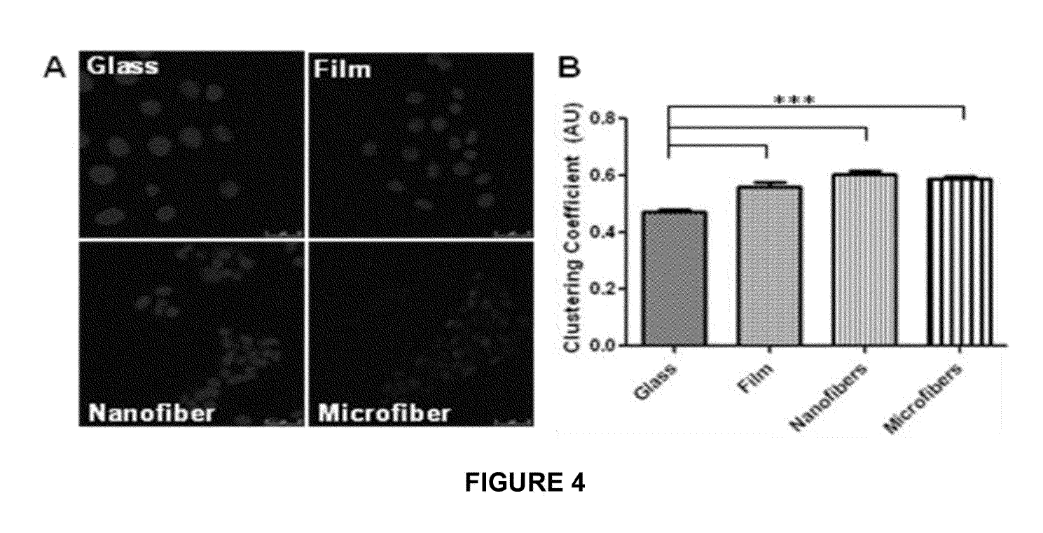

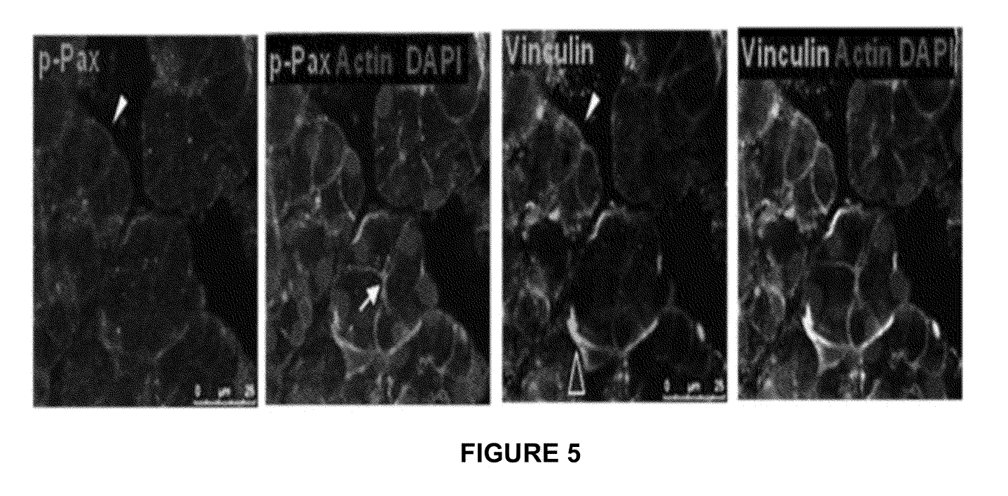

There are many factors within the extracellular environment that influence cell behavior and function, one of which is the nanotopography of the surface material [9-11]. The present inventors observed that nanotopography promotes formation of significantly different cell-surface attachments in salivary gland epithelial cells than do either flat surfaces or microfiber surfaces [5]. Cells seeded upon 2D glass or other non-natural hard surfaces typically form focal adhesions [1,2] at substrate attachment points, whereas most epithelial cells cultured on 3D matrices and in vivo do not produce similar focal adhesion structures [1,3]. Salivary cells seeded on PLGA nanofiber surfaces, for example, do not form focal adhesions to the same extent as cells seeded on non-natural, flat, hard substrates, such as glass or PLGA flat films (FIG. 4.) The pattern of focal adhesion proteins in cells seeded on nanofibers is similar to their appearance in adult acinar structures (see FIG. 5.) In FIG. 5, confocal images of adult SMG tissue fragments immunostained for focal adhesion proteins, active phosphorylated (p)-paxillin (green), or vinculin (cyan, and co-stained with actin (red) and DAPI (blue), show low levels of membrane localization of FA proteins in acinar units (white arrowheads). Actin (red) accumulates apically. Other actin-positive, non-acinar cells show strong staining of p-pax and vinculin. The pattern of focal adhesion proteins in adult acinar structures is similar to their appearance in salivary gland cells seeded on nanofibers. 63× magnified image scale bars=25 μm.

Additionally, the cells seeded on nanofibers self-organize into rounded clusters similar to cells in vivo and on 3D matrices mimicking the in vivo environment, while those on flat or microfiber substrates are more spread [5] and have a lower clustering coefficient (FIG. 4) [14]. Generally, the more in vivo-like morphology of cells cultured on nanofibers supports the hypothesis that the nanofiber diameter influences cell differentiation.

### Nanofibers

Electrospun nanofibers have been utilized in many biomedical applications as biomimetics of extracellular matrix proteins that promote self-organization of cells into 3D tissue constructs. To date, however, there have been few reports of nanofiber structures capable of supporting epithelial cell growth and differentiation.

Nanofiber features of the cell support of the invention are prepared using biocompatible materials. Natural polymers such as silk and laminin may be used. Other polymers suitable for use in preparing the cell support of the present invention include but are not limited to poly(∈-caprolactone) (PCL), poly(∈-caprolactone-co-ethyl ethylene phosphate) (PCLEEP), poly(lactic acid) (PLA), poly(lactic-co-glycolic acid) (PLGA), poly(lactic acid-co-∈-caprolactone) (PLACL), and polydioxanone (PDO).

Exemplary non-degradable polymers could also be used, which include poly acrylamide (PAAm), poly acrylic acid (PAA), poly acrylonitrile (PAN), poly amide (Nylon) (PA, PA-4,6, PA-6,6), poly aniline (PANI), poly benzimidazole (PBI), poly bis(2,2,2-trifluoroethoxy) phosphazene, poly butadiene (PB), poly carbonate (PC), poly ether amide (PEA), poly ether imide (PEI), poly ether sulfone (PES), poly ethylene (PE), poly ethylene-co-vinyl acetate (PEVA), poly ethylene glycol (PEG), poly ethylene oxide (PEO), poly ethylene terephthalate (PET), poly ferrocenyldimethylsilane (PFDMS), poly 2-hydroxyethyl methacrylate (HEMA), poly 4-methyl-1-pentene (TpX), poly methyl methacrylate (pMMA), poly p-phenylene terephthalamide (PPTA), poly propylene (PP), poly pyrrole (PPY), poly styrene (PS), polybisphenol-A sulfone (PSF), poly sulfonated styrene (PSS), Styrene-butadiene-styrene triblock copolymer (SBS), poly urethane (PU), poly tetrafluoro ethylene (PTFE), poly vinyl alcohol (PVA), poly vinyl carbazole, poly vinyl chloride (PVC), poly vinyl phenol (PVP), poly vinyl pyrrolidone (PVP), and poly vinylidene difluoride (PVDF).

In some embodiments, “tunable” polymers, that is, polymers that incorporate functional groups which allow chemical and mechanical properties of the polymer to be modified, are used. Mechanical properties such as the rigidity/elasticity of the nanofiber structure may be modulated, for example, by crosslinking within the nanofiber structure, once the nanofibers have been disposed on the surface of the substrate. Polymers can also be modified to include bioactive molecules, for example, components of extracellular matrix (ECM), such as ECM proteins including but not limited to collagen, fibronectin, elastin, laminins and perlecan.

### Formation of Nanofiber Structure

In one embodiment, electrospinning is used to prepare the nanofiber structure of the invention. The process of electrospinning is well known to those of skill in the art. It generally involves the creation of an electrical field at the surface of a liquid. The resulting electrical forces create a jet of liquid which carries electrical charge. The liquid jets may be attracted to other electrically charged objects at a suitable electrical potential. As the jet of liquid elongates and travels, it will harden and dry. The hardening and drying of the elongated jet of liquid may be caused by cooling of the liquid, i.e., where the liquid is normally a solid at room temperature; evaporation of a solvent, e.g., by dehydration, (physically induced hardening); or by a curing mechanism (chemically-induced hardening). The fibers produced are collected on a suitably located, oppositely charged target collector or substrate.

Typically, the electrospinning apparatus includes an electrodepositing mechanism and a target substrate. The electrodepositing mechanism includes at least one container to hold the solution that is to be electrospun. The container has at least one orifice or nozzle to allow the streaming of the solution from the container. If there are multiple containers, a plurality of nozzles may be used.

One or more pumps (e.g., a syringe pump) used in connection with the container can be used to control the flow of solution streaming from the container through the nozzle. The pump can be programmed to increase or decrease the flow at different points during electrospinning.

The electrospinning occurs due to the presence of a charge in either the orifices or the collector, while the other is grounded. In some embodiments, the nozzle or orifice is charged and the collector is grounded. Those of skill in the electrospinning arts will recognize that the nozzle and solution can be grounded and the collector can be electrically charged. The collector can also be specifically charged or grounded along a preselected pattern so that the solution streamed from the orifice is directed into specific directions. The electric field can be controlled by a microprocessor to create an electrospun matrix having a desired geometry. The collector and the nozzle or nozzles can be engineered to be movable with respect to each other thereby allowing additional control over the geometry of the electrospun matrix to be formed. The entire process can be controlled by a microprocessor that is programmed with specific parameters that will obtain a specific preselected electrospun matrix.

Optimization of Nanofiber Characteristics Using a design of experiment approach, the system and process parameters of the electrospinning process can be optimized concurrently, and their effects on the diameter of the resulting fibers computed into a single model. A transfer function is used to reproducibly produce nanofibers of a defined diameter, which can then be confirmed by imaging with a scanning electron microscope (see Jean-Gilles et al. Novel Modeling Approach to Generate a Polymeric Nanofiber Scaffold for Salivary Gland Cells. Journal of Nanotechnology in Engineering and Medicine. 1(3): 031008. doi:10:1115/1.4001744)

### Formation of Curved Nanofiber Structures

In one embodiment, a substrate having a concave surface upon which the nanofibers are to be disposed takes the form of a micro-patterned crater array. The arrays are prepared as discussed above, for example by spincoating polydimethylsiloxane (PDMS) onto a silicon master made using standard lithography processing techniques. The PDMS is allowed to cure and then the mold is peeled from the silicon master.

The nanofibers are then electrospun or otherwise generated from a polymer solution, for example, PLGA, in an appropriate solvent, to generate randomly-oriented nanofibers having a diameter in the range of about 50 nm to about 1000 nm disposed over the PDMS features; in one embodiment, nanofibers will have a diameter in the range of about 100-500 nm, in another embodiment, nanofibers will have a diameter in the range of 150-250 nm. The nanofiber structure has an appropriate thickness when it completely covers the substrate, so as to isolate any cells cultured on the nanofibers from any effects of the substrate material itself.

After the nanofibers have been deposited on the substrate, the nanofiber features are soaked in a physiologically appropriate media or buffer solution for a period of time sufficient to ensure that the nanofiber structure has conformed to the curvature of the craters in the substrate, from a few hours to several days. In one embodiment, nanofiber structures are soaked for about 1-5 days.

### Pore Size

Nanofibers (average diameter ˜250 nm) were imaged using a scanning electron microscope (SEM) and pore size measurements were obtained using the microscope's integrated annotation software. Distances between fibers on the same Z-plane were measured across samples and average pore sizes were estimated. Using 20 measurements, pore sizes ranged between about 1.85 μm and about 3.39 μm with an average pore size of about 2.51±0.52 μm, which is small enough so that cells, which are typically between 5 and 10 μm in size, will not fall below the top layer of fibers.

### Z-Thickness of Nanofiber Mats

The thickness, or height in the Z-direction, of nanofibers (average diameter ˜250 nm) was measured using cross-sectional SEM. Ten samples were imaged, and the thicknesses of the fiber mats were determined using the SEM's integrated annotation software. A range of thickness between about 0.71 μm and 1.63 μm and an average value of about 1.16±0.31 μm was obtained, which is more than enough distance so that the cells growing on top of the surface do not contact the substrate beneath.

### Biochemical Modification of the Nanofiber Scaffold

Unmodified nanofiber scaffolds provide signals to salivary gland epithelial cells to both proliferate and to initiate apicobasal polarity in culture. These signals were enhanced by covalent coupling of chitosan and/or laminin-111 to the nanofibers. In some embodiments, electrospun nanofiber scaffolds are modified by the addition of basement membrane components, such as proteins and/or polysaccharides, for example, laminin and chitosan, respectively. More information regarding selective functionalization of nanofiber scaffolds to regulate secretory epithelial cell proliferation and polarity can be found in Cantara et al. Biomaterials 33:8372-8382 2012; the contents of which are hereby incorporated by reference.

### Cell Culture on Nanofiber Scaffold

Secretory epithelial cells, either primary cells obtained from an appropriate source or a non-transformed epithelial cell line (for example, a cell line that, although immortalized, retains the differentiated properties of a polarized ductal epithelial cell) are then cultured and seeded on the cell support. Examples of primary cells for culture on the cell support of the invention include but are not limited to human or other mammalian secretory epithelial cells, for example, salivary gland epithelial cells, lacrimal gland epithelial cells, exocrine pancreas epithelial cells, sweat gland epithelial cells and the like. Primary embryonic salivary gland cells can be obtained as described in Wei, et al 2007.

Established cell lines which are suitable for growth and development on the cell support of the present invention include but are not limited to: SIMS (an immortalized submandibular salivary gland epithelial cell line derived from a 22 day old mouse; see Laoide et al., Immortalised mouse submandibular epithelial cell lines retain polarized structural and functional properties. Journal of Cell Science 109:2789-2800 1996; incorporated by reference), HSG cells (an immortalized human salivary gland cell line) sca9 (a mouse salivary gland tumor cell line), parc10 (an immortalized rat parotid salivary gland acinar cell line), smgc10 (an immortalized rat submandibular gland cell line) cells, and HSG (a neoplastic salivary gland epithelial ductal cell line).

SCA9 cells are cultured tumor cells of urine submandibular gland origin and suitable for use in conjunction with the present invention as are ParC10 cells, an SV40 immortalized rat parotid acinar cell line, SMGC10, an SV40 immortalized rat submandibular acinar cell line and HSG, a neoplastic epithelial duct cell line established from an irradiated human salivary gland. (see Eur J Oral Sci. 2000 February; 108(1):54-8. Cultured tumor cells of murine submandibular gland origin: a model to investigate pHi regulation of salivary cells. Trzaskawka E, Vigo J, Egea J C, Goldsmith M C, Salmon J M, De Periere D D; Quissell, D. O., K. A. Barzen, R. S. Redman, J. M. Camden, and J. T. Turner. Development and characterization of SV40 immortalized rat parotid acinar cell lines. In Vitro Cell. Develop. Biol. 34: 58-67, 1998; Quissell, D. O., K. B. Barzen, D. C. Gruenert, R. S. Redman, J. M. Camden, and J. T. Turner. Development and characterization of SV40 immortalized rat submandibular acinar cell lines. In Vitro Cell. Dev. Biol. 33: 164-173, 1997; Shirasuna, K. Sato, M., and Miyazaki, T. A neoplastic epithelial duct cell line established from an irradiated human salivary gland. Cancer (Phila.), 48:745-752, 1981.

In one embodiment, primary submandibular cells or other secretory epithelial cells are prepared using a tissue explant culture method, as previously described for mouse cells, Wei, 2007 et al. and human cells (for example, see Joraku et al., In-vitro reconstitution of three-dimensional human salivary gland tissue structures. Differentiation. 2007; Joraku et al., Tissue engineering of functional salivary gland tissue. Laryngoscope. 2005, the disclosures of which are hereby incorporated by reference). Briefly, normal human or mammalian exocrine gland tissue samples are obtained and cut into 1 mm-sized fragments, plated on culture dishes, and placed in serum-free keratinocyte growth medium containing 5 ng/ml epidermal growth factor (EGF) and 50 μg/ml bovine pituitary extract (BPE). The cells are incubated and grown in a humidified atmosphere chamber containing 5% CO₂ and maintained at 37° C. Monolayer cells are trypsinized, and single-cell suspensions prepared for seeding onto a cell support of the invention.

Other methods for the culture of human salivary gland and other endoderm-derived epithelial tissues, such as lung and pancreas are known to those of skill in the art (see, e.g., Tran et al., 2005, Mondrinos et al., 2006 and Harris and Coleman, 1987).

In an alternate embodiment, tissue is completely dissociated rather than cut into pieces, and other media supplements, such as insulin, transferring, and selenium, which are used for culture of many other primary cells are included in the growth medium.

Cells are evaluated for viability, and a suspension of viable cells in an appropriate cell culture medium is prepared for seeding onto the cell support. Cells are seeded at a density in the range of about 1,000 to 100,000 cells per well of a tissue culture plate into which a cell support of the invention has been placed; in one embodiment, cells are seeded at a density in the range of about 10,000 to 50,000 cells per well and cultured for 24 hours or more prior to evaluation of differentiation marker expression using various assays, such as immunocytochemistry.

### Indicators of Differentiation and Morphogenesis

Cells are monitored for progress of differentiation and morphogenesis using appropriate differentiation markers and conventional microscopic techniques, including confocal, and scanning electron microscopy. Two well-characterized proteins indicative of acinar differentiation are α-amylase, an enzyme found primarily in serous secretions, and aquaporin-5 (Aqp-5), the major transmembrane water channel protein found on the apical, or lumen-facing, side of the plasma membrane of acinar cells. α-amylase is a good marker for salivary gland differentiation, as the enzyme is primarily only produced by the salivary gland and the pancreas.

Aquaporin-5 has been shown to be present in serous and mucous acini of the salivary glands, as well as in the lacrimal glands which lubricate the eye, alveolar epithelial cells of the lung, and corneal epithelial cells within the eye. Aqp-5 is only considered an acinar differentiation marker. Aqp-5 expression is crucial to the secretion of saliva by acini. Additionally, it is important that Aqp-5 be located on the apical surface of the cells.

Another more generalized indicator of differentiation is the formation of tight junctions between epithelial cells. Two common tight junction proteins which are well-characterized in salivary glands and considered important markers in determining salivary gland tight junction formation are occludin and the intracellular linking protein zonula occludens-1 (ZO-1).

Another cell-cell junction protein which is important in forming tight junctions between cells is E-cadherein. Like ZO-1, E-cadherein is found in the adherens junction of epithelial cells, but like occludin, is a transmembrane protein. Although E-cadherin is primarily found in the adherens junction, it has been shown to be important in both the formation and regulation of tight junctions.

The expression of various developmental markers is determined by immunocytochemistry of cultured cells or tissues in accordance with methodology know to those of skill in the art. In an embodiment in which murine epithelial cells have been cultured on the cell support, for example, immunocytochemistry is performed as previously described (Daley et al. 2009; Larsen et al. 2003). Briefly, crater arrays containing the cell support(s) of the invention are incubated in blocking solution (20% donkey serum (Jackson ImmunoResearch) containing Mouse on Mouse (M.O.M.) blocking reagent (Vector Laboratories, Burlingame, Calif., USA) in 1×PBS containing 0.05% Tween 20 (PBS-T)). Primary antibodies are diluted in an antibody diluent consisting of 10% bovine serum albumin (BSA, Sigma-Aldrich Corp., St. Louis, Mo., USA) in phosphate-buffered saline (PBS) (PBSA) and incubated overnight at 4° C. Cell preps are washed 4×10 min in 1×PBS-T after each antibody incubation step. Cyanine dye-conjugated AffiniPure F(ab′)₂ fragment secondary antibodies are diluted 1:100 in diluent and incubated with the tissue overnight at 4° C. Following the final wash sequence constructs are mounted using glass coverslips with FLUORO-GEL Gel-Mount (Electron Microscopy Sciences) containing 1 mg/mL p-phenylene-diamine (PPD) antifade reagent. Constructs are imaged on a confocal microscope. All images are captured using identical settings. Negative controls include incubations with secondary antibody only.

For high throughput screening, laser scanning confocal microscopy may be used. Cells are evaluated for, inter alia, development of apical-basal polarity and columnar morphology and apical localization of a marker for tight junctions which are an apical cell-cell adhesion structure that is required for formation in of a polarized cell monolayer. At an appropriate stage of development, cells may be used for high throughput screening or other testing.

The present invention also provides a device and method for screening compounds that modulate secretory epithelial cell development. The device comprises a substrate having a concave surface, a nanofiber structure disposed over the concave surface of the substrate and secretory epithelial cells seeded on the nanofiber structure of the device. The method comprises providing a device comprising a substrate having a concave surface and a nanofiber structure disposed over the concave surface of the substrate, seeding secretory epithelial cells on the nanofiber structure of said cell support; incubating said cell support and cells under conditions otherwise sufficient for proliferation and/or differentiation to occur in the presence and absence of the compound to be tested; monitoring development of cells in the presence of the compound and monitoring development of cells in the absence of the compound and comparing the development of cells grown in the presence of the compound with the development of cells grown in the absence of the compound to determine the effect of the test compounds on development, proliferation, differentiation and morphogenesis.

Identification of a test compound that modulates development of secretory epithelial cells is made by evaluating various parameters of development of the cells seeded on the cell support; parameters to be evaluated include cell morphology of the cells seeded on the nanofiber structure; expression of differentiation markers such as ZO-1; production of organ product such as proteins found in saliva or pancreatic fluid or any combination of these parameters.

It should be appreciated that the invention should not be construed to be limited to the examples that are now described; rather, the invention should be construed to include any and all applications provided herein and all equivalent variations within the skill of the ordinary artisan.

### EXAMPLES

### Example 1

### Electrospinning of Polylactic-Co-Glycolic Acid Nanofiber Structures

Poly(D-lactide-co-glycolide) (PLGA), with a lactic to glycolic acid ratio of 85:15 and a molecular weight of 95,000 Da, was purchased from Birmingham Polymers (Pelham, Ala.). Hexafluoroisopropanol (HFIP), dimethylformamide (DMF), sodium chloride (NaCl), and magnesium sulfate (MgSO₄) were purchased from Sigma-Aldrich USA (St. Louis, Mo.). An automatic syringe pump was purchased from New Era Pump Systems Inc. (Wantagh, N.Y.). 3 ml syringes were purchased from Becton, Dickinson and Company (Franklin Lakes, N.J.). Needles with an inner diameter of 0.25, 0.41 and 0.51 mm were purchased from EFD Inc. (East Providence, R1). Polytetrafluoroethylene (PTFE) tubing was purchased from VWR International (West Chester, Pa.). All chemicals were used as supplied without further purification.

### Electrospinning Setup

Methods and devices for electrospinning are well known in the art. To electrospin a nonwoven mat of nano- and micro-scale fibers, PLGA was dissolved in HFIP or DMF and loaded into a 3 ml syringe. The syringe was loaded into an automatic syringe pump, and PTFE tubing was used to shuttle the polymeric solution from the syringe to a metal needle tip. The needle was suspended vertically over a grounded aluminum collector plate at a fixed distance, and a voltage supply was wired to the metal needle with an alligator clip. The distance between the needle tip and the collector is labeled as the “spinneret-to-collector” distance in all of the figures and tables.

### PLGA-DMF Fibers

PLGA was dissolved in N,N-dimethylformamide (DMF) solvent at concentrations ranging from 30-35% (w/v) and stirred in a closed container for 18 hours at room temperature to create a homogenous solution, as reported previously [36]. The pumping rate for PLGA-DMF experiments was between 6 and 8.5 μl min⁻¹ and the needle inner diameter was 0.41 mm. A positive DC potential ranging from 15 to 20 kV was applied at a spinneret-to-collector distance ranging from 15 to 20 cm.

### PLGA-HFIP Fibers

Next PLGA was dissolved in 1,1,1,3,3,3-hexafluoro-2-propanol (HFIP) solvent at concentrations ranging from 4 to 18% (w/w), and stirred in a closed container for 18 hours at room temperature. The pumping rate for PLGA-HFIP experiments was between 3 and 25 μl min⁻¹ and the needle inner diameter ranged from 0.25 to 0.51 mm. The electric potential difference applied ranged from 8 to 25 kV and the distances between the needle tip and the collector ranged from 5 to 15 cm. To eliminate HFIP-induced damage to the tube and connectors, metal luer connectors and polytetrafluoroethylene (PTFE) tubing were used. Using a PLGA concentration of 4% (w/w) in HFIP with 1% NaCl, nanofibers with an average diameter of 86.1 nm±17.2 nm were attained.

### Nanofiber Modifications

In some embodiments, electrospun PLGA fiber scaffolds were modified with either FITC-chitosan or laminin-111 prior to cell seeding. PLGA fiber scaffolds were hydrolyed by incubation in a 50 mM NaOH solution for 30 min. at room temperature. Then scaffolds were activated by incubating in 4 mM 1-ethyl-3-(3-dimethylaminopropyl)carbodiimide (EDAC) (Sigma Aldrich, St. Louis Mo.), 100 mM N-hydroxysuccinimide (NHS), 10 mM 2-(N-morpholino)ethanesulfonic acid (MES) solution for 1 hour at room temperature. These chemical treatments did not significantly affect the fiber macrostructure, as determined by SEM (data not shown). Next, scaffolds were incubated in either a 1% FITC-chitosan solution in 0.1M acetic acid or a 10 μg/ml laminin-111 solution in cold 1×PBS at 4 C overnight. To confirm attachment of FITC-chitosan, confocal images were acquired. To confirm attachment of laminin-111, fiber scaffolds were immunostained with anti-laminin antibody and observed with confocal microscopy. All scaffolds were washed and incubated in sterile 1×PBS supplemented with 1× penicillin-streptomycin, and incubated in sterile cell culture media prior to use in experiments.

### Scanning Electron Microscopy (SEM) Characterization of Nanofibers

Fiber diameter and general morphology were characterized using a 1550 field emission scanning electron microscope (SEM) (Leo Electron Microscopy Ltd, Cambridge, UK; Carl Zeiss, Jena, Germany). Samples were mounted on 1 cm² stubs using carbon tape and were sputter-coated with ˜5 nm gold-palladium to avoid charging the sample. Images were collected at a working distance of 3 mm and with an acceleration gun voltage of 2 kV. Using the incorporated digital annotation software, diameter measurements (15 per sample) were acquired, and arithmetic means and standard deviations were calculated for all fibers.

### Statistical Analyses and Surface Plots

All statistical analyses were performed using the statistical software package MINITAB. Design of Experiment (DOE) analyses were performed using a response surface design with non-coded values. The data was analyzed by mean of regression analysis. The R-squared values were greater than 0.95. Within this dataset, none of the system and process parameters evaluated were found to be confounded. Factors with a p-value greater than 0.05 were excluded. The exclusion of factors that failed the null hypothesis yielded the transfer functions, which were further optimized using MINITAB's optimizer tool and Microsoft Excel®' solver tool.

After using the transfer function for PLGA-HFIP fibers with 1% NaCl to solve for appropriate system and process parameters needed to electrospin nanofibers (diameter 250 nm) and microfibers (diameter 1200 nm), fibers of each diameter were prepared on 12 mm coverslips. A thin layer of PLGA was solvent cast using 60 ml of 3% PLGA (w/w) dissolved in HFip and dried for 1 h at room temperature immediately before electrospinning in order to create an adhesive layer between the glass and the fibers and to prevent fiber detachment during culture. For nanofibers and microfibers, samples were electrospun for 45 min and 10 min, respectively. Vectabond-coated glass coverslips without fibers and flat PLGA polymer-coated glass coverslips were used as flat surface and material controls, respectively. PLGA material control films were generated by spin-coating 80 ml of 5% PLGA (w/w) dissolved in chloroform onto Vectabond-treated glass coverslips for 1 min at 1500 rpm, and then placed on a hotplate at 200° C. until complete solvent evaporation occurred. All surfaces were sterilized by UV irradiation for at least 1 hr, pre-soaked in sterile phosphate buffered saline (PBS) for 48 hrs, followed by 24 hours in complete cell media before cell culture.

### Example 2

### Cell Culture, Viability Assays and SEM Characterization of Cells on Nanofibers

SIMS, an immortalized submandibular salivary gland epithelial cell line derived from a 22 day old mouse [20, 24], was cultured in complete cell media, Dulbecco's Modified Eagle Medium (DMEM), 10% fetal bovine serum (FBS), and 100 U/ml penicillin-streptomycin, as previously described (all Invitrogen, Carlsbad, Calif.).

PLGA fibers were electrospun onto 12 mm glass coverslips coated with Vectabond (Vector Laboratories), as per manufacturer's protocols, and placed into individual wells in a 24-well tissue culture plate. Vectabond-coated glass coverslips without fibers and flat PLGA polymer-coated glass coverslips were used as flat surface and material controls, respectively. Approximately 50 μl of 3% (w/v) PLGA in HFIP was added to each coverslip and air-dried for 24 hrs at room temperature to create the flat PLGA coatings. All surfaces were sterilized by UV irradiation for at least 1 hr, pre-soaked in sterile phosphate buffered saline (PBS) for 48 hrs, followed by 1 hr in complete cell media before cell culture.

For trypan blue viability and cell proliferation assays, SIMS cells (10,000 cells/well) were seeded in complete media. At the indicated intervals, coverslips were removed, transferred to fresh plates, prior to cells being detached by mild trypsinization, treated with trypan blue, and manually counted using a hemocytometer. For fluorescent live-dead assays (Invitrogen), SIMS cells were seeded (10,000 cells/well) on different surfaces as indicated and cultured for 24 hrs in complete media. Cells were washed in PBS to remove media and incubated with a mixture of calcein (1 μM), to detect live cells and ethidium homodimer-1 (2 μM) to label dead cells for 20 mins. Images were obtained using an inverted fluorescent microscope (CellObserver Z1, Zeiss, Jena, Germany) with a 20× objective at excitation wavelengths of 488 nm (to detect the fluorescent biproduct of calcein metabolism) and 543 nm (to detect ethidium).

For SEM, approximately 3×10⁴ cells/well were seeded onto coated or non-coated 12 mm coverslips and allowed to adhere overnight to different surfaces before fixing and processing for SEM. Briefly, cells were fixed with 3% glutaraldehyde solution in 0.1M phosphate buffer containing 0.1M sucrose for 2 hrs at room temperature. The samples were then rinsed three times in PBS, dehydrated in a graded ethanol series and slowly infiltrated with hexamethyldisilazane (HMDS) for drying. SEM characterization of the samples was done using an FEI Nova NanoSEM 600 field emission scanning electron microscope (FEI Company, Hillsboro, Oreg.). Uncoated samples were mounted on 1 cm² stubs using carbon tape and imaged in low vacuum mode with water vapor introduced into the specimen chamber to minimize sample charging. An extraction voltage of 4 kV and a working distance of 5 mm was used for imaging.

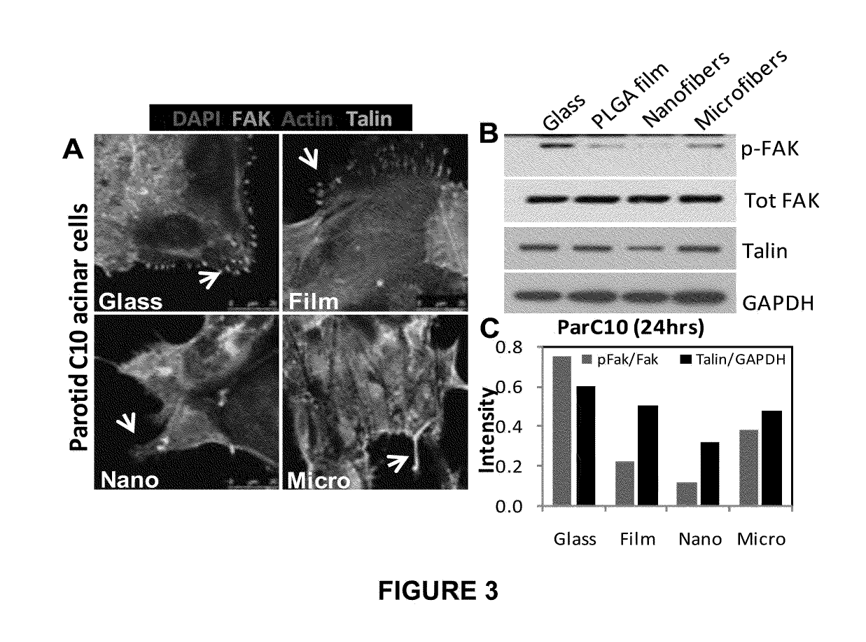

Hexafluoroisopropanol (HFIP) was selected as an alternate solvent due to its low boiling point of 58° C., and dielectric constant of 16.70 at 20° C., which is nearly half of that of DMF. Due to the drastic difference in boiling point, a much lower potential can be used to electrospin the nanofibers using HFIP as a solvent as compared to fibers produced when using DMF as a solvent [42]. When fibers were electrospun from a PLGA-HFIP solution, a positive linear relationship was observed, as reported in previous studies [40]. Although beading was reduced with increased PLGA concentration, beading was still prevalent in all samples regardless of solution concentration (FIG. 3a, b, c). To generate a reliable transfer function for electrospinning PLGA nanofibers of uniform morphology, additional changes to the properties of the solution were required.

Previous reports have indicated that the presence of salts can eliminate beading from electrospun nanofibers [36]. We evaluated sodium chloride (NaCl) and magnesium sulfate (MgSO₄), at 1 wt % in each solution, for their effect on nanofiber uniformity when HFIP was used as a solvent. Both salts reduced the total number of beads observed; however NaCl eliminated beads much more effectively than MgSO₄. The addition of NaCl to an 8.5 wt % solution yielded fiber diameters that ranged from an average of 110 nm to 278 nm, whereas addition of MgSO₄ yielded fibers whose average diameters ranged from 315 nm to 379 nm. Additionally, the diameter distribution is significantly less with NaCl than it is with MgSO₄ under specific conditions. These effects were also observed where addition of NaCl resulted in fibers of a more consistent diameter than those generated in the presence of MgSO₄. Since NaCl expanded the range over which bead-free nanofibers were produced, all subsequent fibers were prepared using 1 wt % NaCl in HFIP solvent, and the subsequent transfer function generated by Minitab for HFIP used DOE experimental runs with 1% NaCl in solution.

Using HFIP and 1 wt % NaCl, a DOE-based approach was again used to design a series of experiments to assess the effects of varying both system and process parameters on nanofiber diameter. A response surface regression was performed to compare the effects of PLGA concentration, the potential, the feeding rate and the spinneret-to-collector distance on the average diameter of the fibers. Another process parameter, the diameter of the needle tip, was controlled and kept constant at 0.51 mm. The analysis was performed using non-coded units and the regression coefficients were evaluated. As previously observed with DMF, the concentration has the strongest positive correlation with the average diameter of the fibers. An increase in the potential and the spinneret-to-collector distance negatively affected the diameter of the fibers, while the feeding rate showed the weakest correlation to the average diameter of the fibers.

Since the PLGA concentration had the strongest positive effect on the average diameter of the fibers, its effect in concert with the other parameters (potential, spinneret-to-collector distance, feeding rate) was evaluated. In general, as the concentration of PLGA increased along with the other parameters, the diameter of the fibers appears increased. However, at low PLGA concentrations, the other process parameters have a stronger influence than at high PLGA concentrations. When the value of the process parameters are increased above a certain threshold, the concentration of the PLGA must be increased significantly to overcome the irregularity in the diameter of the fibers. This understanding allows for the modulation of both parameters in parallel in order to change the diameter of the fibers without the need for PLGA concentrations as high as those used with DMF as the solvent.

To facilitate synthesis of nanofibers of a defined diameter, we performed a regression analysis of these data to produce a transfer function for the average diameter of the nanofibers electrospun in HFIP:

dₐᵥₑ=161X₁−308X₂+0.1X₃−71.2X₄−3078

Equation 1 gives the transfer function of the average diameter (dₐᵥₑ) of fibers electrospun from a solution of PLGA in HFIP and 1% NaCl. The Independent variables are PLGA concentration (X₁), the potential (X₂), the feeding rate (X₃), and the spinneret-to-collector distance (X₄).
Validation of the Transfer Function for PLGA-HFIP Solution with 1% NaCl

To validate the transfer function, two target fiber diameters were selected: 250 nm (nanofibers) and 1200 nm (microfibers), and the equation was solved for these two values to provide settings for each of the process and systems parameters. We set standard deviations of 50 nm and 200 nm as acceptable for each fiber diameter, respectively. Using the parameters specified by the transfer function, we generated nanofibers by electrospinning and measured the diameters of the resulting fibers using SEM. We obtained beadless nanofibers of an average diameter of 247.2 nm and 1276.5 nm, under the two conditions, which fell within the acceptable limits we set for thin and thick fibers, respectively. These data validate the applicability and effectiveness of the transfer function for producing uniform PLGA nanofibers of a defined diameter.

### Utility of the Transfer Function in the Preparation of Specified PLGA Nanofiber Diameter for Use as Salivary Gland Epithelial Cell Scaffold

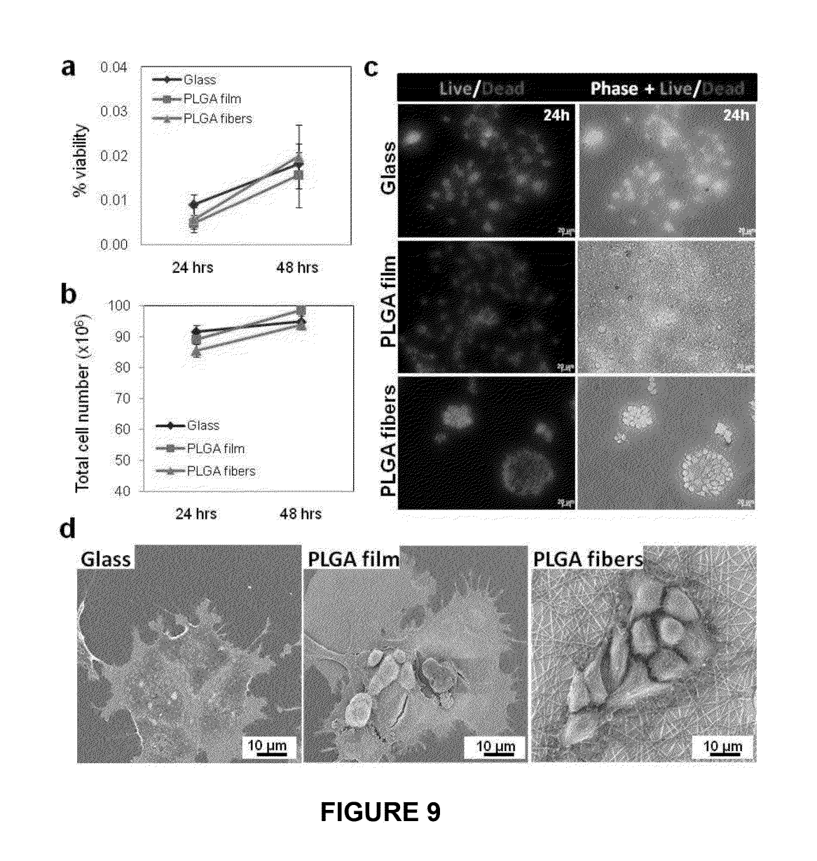

To assess the suitability of the 250 nm nanofibers as a substrate for salivary gland cells, we used the SIMS mouse submandibular gland cell line as a model cell. To determine if the salivary gland cells could attach to the nanofibers produced during this study, we compared the overall viability and morphology of SIMS salivary gland epithelial cells seeded on glass (a typical substrate for in vitro cell culture) and a PLGA film (a material control) to those seeded on the PLGA nanofibers (247.2 nm mean diameter). To examine cell proliferative ability, SIMS cells were cultured on flat glass, flat PLGA film, or PLGA nanofibers for up to 48 hrs, and cells were counted at 24 hr intervals. Cells proliferated comparably on all three substrates, and perhaps slightly better on PLGA nanofibers than on glass or PLGA films (FIG. 9), indicating that the cells could at least proliferate as well on nanofibers as on flat controls. To account for possible differences in cell viability on the different surfaces, trypan blue staining was used in cell counting experiments to exclude non-viable cells, but significant differences in cell viability on the different substrates were not apparent (FIG. 9b). As an additional confirmation of cell viability, the cells were exposed to calcein AM, which is metabolized to produce a fluorescent green metabolite in live cells, and ethidium homodimer-1, which is non-specifically absorbed only by dead cells and fluoresces red. Few ethidium-positive cells were detected under any condition, and yet almost all cells fluoresced green, indicative of a viable cell population (FIG. 9c).

While the cells seeded upon glass or flat PLGA surfaces generally grew as single dispersed cells, SIMS cells seeded upon the PLGA nanofibers self-organized into cellular aggregates suggestive of an acinar cell cluster (FIG. 9c). To characterize cell aggregate morphology further, SIMS cells were again seeded on both flat surfaces and on the nanofibers, grown for 18 hrs, fixed, and imaged using scanning electron microscopy SEM (FIG. 9d). The cells seeded on the flat surfaces flattened and spread significantly, characteristic of cells in a 2D monolayer, while the SIMS cells grown on nanofibers attached to the fibers and assumed a more three-dimensional, rounded morphology, more typical of salivary gland cells in vivo. The cells did not migrate between the nanofibers, but instead remained on top of the nanofiber scaffold. These data indicate the nanofibers facilitate self-organization of salivary gland cells into a more desirable 3D morphology than is observed on flat substrates.

Cell Behavior when Grown on Curved Nanofiber Structure

To assess the suitability of the 250 nm nanofibers as a substrate for salivary gland cells, SIMS, a mouse submandibular gland cell line were used as a model cell. To determine if the salivary gland cells could attach to the nanofibers produced during this study, the overall viability and morphology of SIMS salivary gland epithelial cells seeded on glass (a typical substrate for in vitro cell culture) and a PLGA film (a material control) were compared to those seeded on the PLGA nanofibers (247.2 nm mean diameter). To examine cell proliferative ability, SIMS cells were cultured on flat glass, flat PLGA film, or PLGA nanofibers for up to 48 hrs, and cells were counted at 24 hr intervals. Cells proliferated comparably on all three substrates, and perhaps slightly better on PLGA nanofibers than on glass or PLGA films (FIG. 9a), indicating that the cells could at least proliferate as well on nanofibers as on flat controls. To account for possible differences in cell viability on the different surfaces, trypan blue staining was used in cell counting experiments to exclude non-viable cells, but significant differences in cell viability on the different substrates were not apparent (FIG. 9b). As an additional confirmation of cell viability, the cells were exposed to calcein AM, which is metabolized to produce a fluorescent green metabolite in live cells, and ethidium homodimer-1, which is non-specifically absorbed only by dead cells and fluoresces red. Few ethidium-positive cells were detected under any condition, and yet almost all cells fluoresced green, indicative of a viable cell population (FIG. 9c).

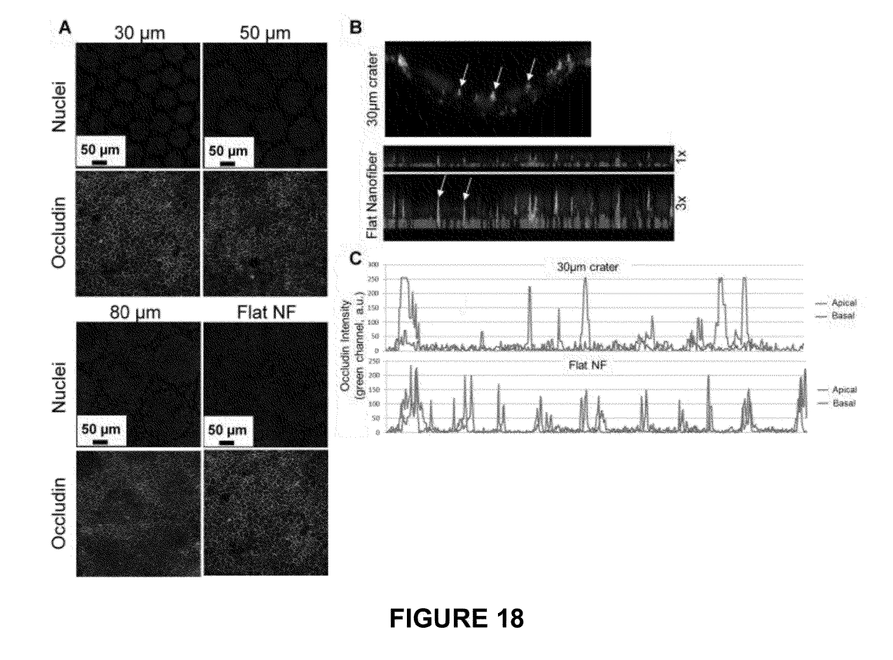

Apical but not localization of the tight junction marker occludin was also observed in SIMS cells cultured in the 30 μm craters but progressively less on the larger craters and flat scaffolds (FIG. 18) as further evidence of apicobasal polarization of the cells.

While the cells seeded upon glass or flat PLGA surfaces generally grew as single dispersed cells, SIMS cells seeded upon the PLGA nanofibers self-organized into cellular aggregates suggestive of an acinar cell cluster (FIG. 9c). To characterize cell aggregate morphology further, SIMS cells were again seeded on both flat surfaces and on the nanofibers, grown for 18 hrs, fixed, and imaged using scanning electron microscopy SEM (FIG. 9d). The cells seeded on the flat surfaces flattened and spread significantly, characteristic of cells in a 2D monolayer, while the SIMS cells grown on nanofibers attached to the fibers and assumed a more three-dimensional, rounded morphology, more typical of salivary gland cells in vivo. The cells did not migrate between the nanofibers, but instead remained on top of the nanofiber scaffold. This indicates that the nanofiber structure facilitates self-organization of salivary gland cells into a more desirable 3D morphology than is observed on flat substrates.

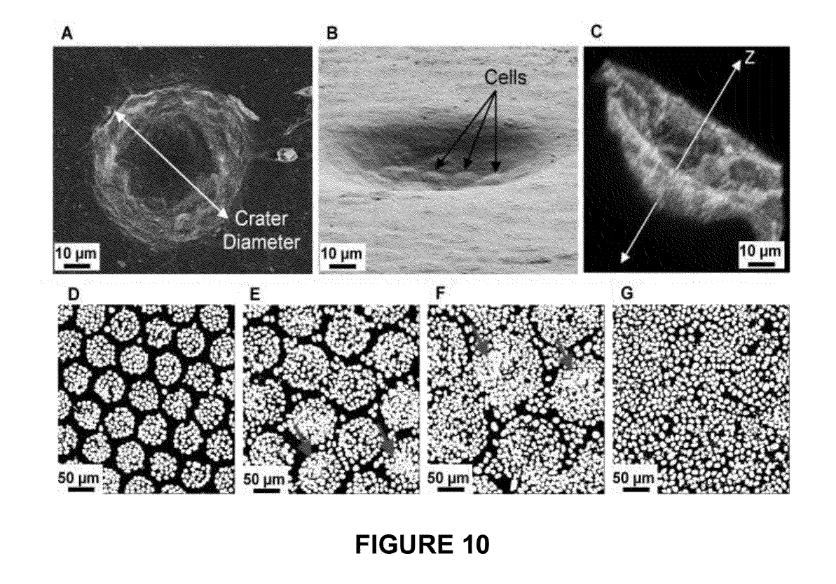

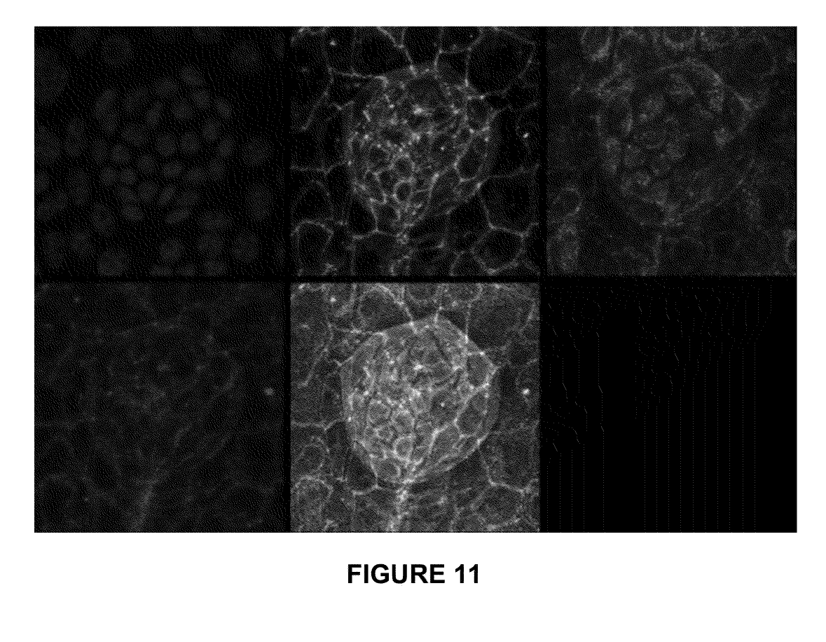

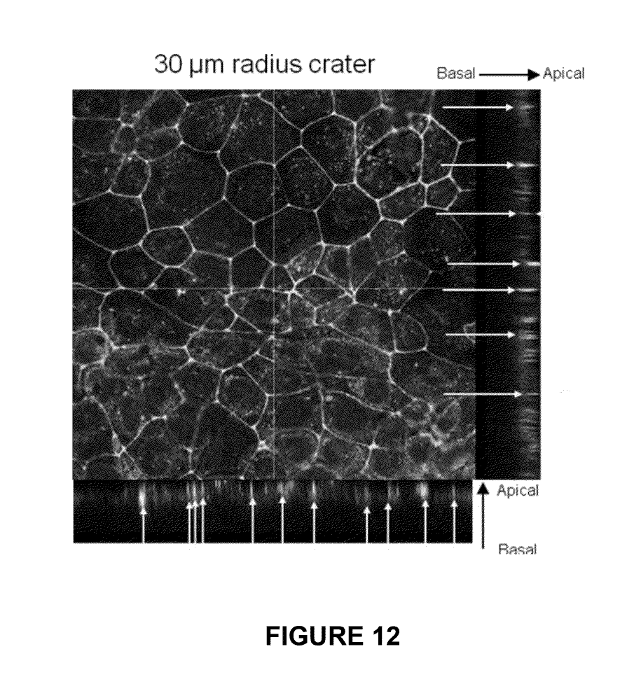

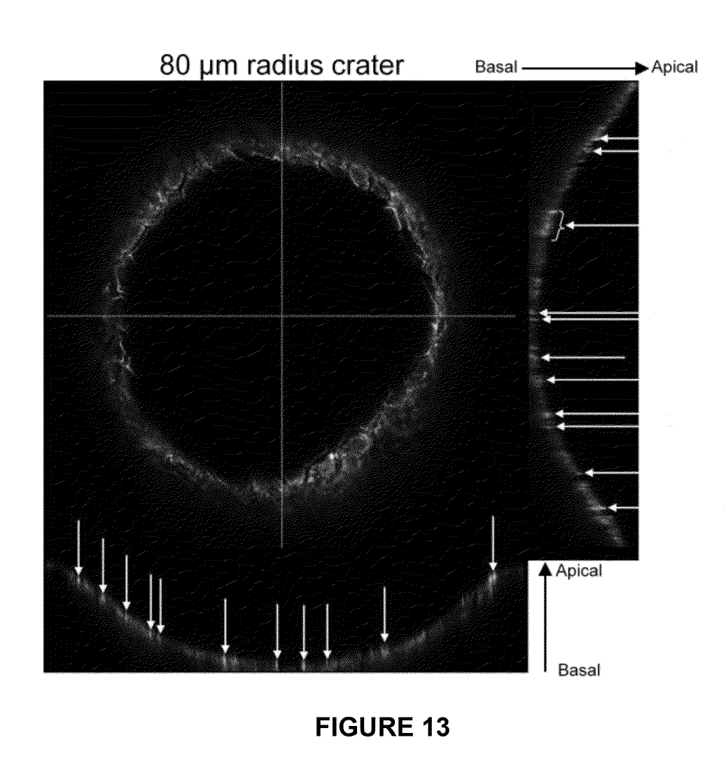

Next, cells were grown on cell supports in which the substrate was flat or patterned with craters having a radius of 30 μm, or 80 μm (and a depth of about 30 μm). The presence and localization of various markers were determined after 96 hours, including ZO-1, a marker for the presence of tight junctions. Higher total ZO-1 expression could be seen in cells in the craters (FIGS. 12 and 13) as compared to cells on the flat surface outside of the crater circumference (FIG. 11). Additionally, the cells growing in the craters exhibited smaller nuclear size suggesting less cell spreading (FIG. 10).

When the cells growing in the crater were viewed from a cross-sectional perspective, ZO-1 appeared as “dots” of stain located at the apical side of the cells in the craters, indicating appropriate apical ZO-1 localization (FIG. 13). As crater size increased (and curvature decreased), ZO-1 staining became more diffuse in the larger craters (FIG. 12), suggesting that, even though fairly well expressed, ZO-1 was no longer localized to the apical portion of the cells, suggesting a lack of mature tight junctions. In comparison, in cells grown on a flat nanofiber control (FIG. 11), ZO-1 staining extends all the way to the basal side of the cells, indicating diffuse localization.

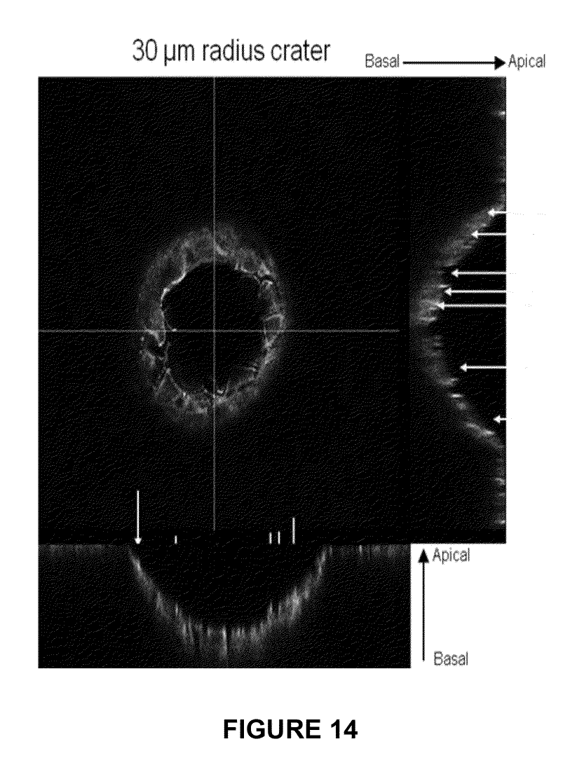

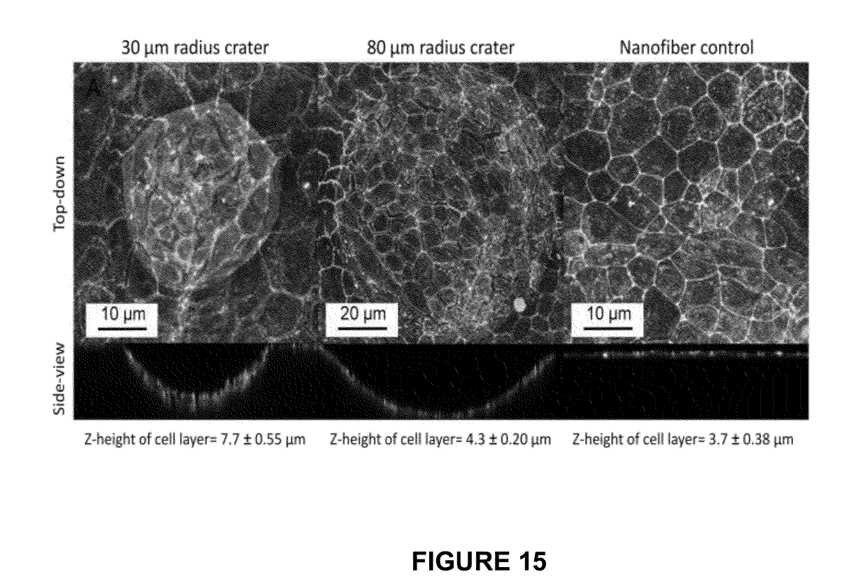

Cell layer Z-height was evaluated for cells grown on surfaces with varying curvature. FIG. 14 shows that the Z-height of the cell layer grown on cells with greater curvature was greater than cells grown on flat surfaces or surfaces with less curvature. As shown in FIG. 15, SIMS Z-height increased with decreasing crater radius.

Cell layer Z-height was evaluated for cells grown on surfaces with varying curvature. FIG. 14 shows that the Z-height of the cell layer grown on cells with greater curvature was greater than cells grown on flat surfaces or surfaces with less curvature. As shown in FIG. 15, SIMS Z-height increased with decreasing crater radius.

### LITERATURE CITED

1. Urquhart, D., and Fowler, C. E. (2006) Review of the use of polymers in saliva substitutes for symptomatic relief of xerostomia. The Journal of clinical dentistry 17, 29-33
2. Shiboski, C. H., Hodgson, T. A., Ship, J. A., and Schiodt, M. (2007) Management of salivary hypofunction during and after radiotherapy. Oral surgery, oral medicine, oral pathology, oral radiology, and endodontics 103 Suppl S66 e61-19
3. Aframian, D. J., Cukierman, E., Nikolovski, J., Mooney, D. J., Yamada, K. M., and Baum, B. J. (2000) The growth and morphological behavior of salivary epithelial cells on matrix protein-coated biodegradable substrata. Tissue Eng 6, 209-216
4. Aframian, D. J., Tran, S. D., Cukierman, E., Yamada, K. M., and Baum, B. J. (2002) Absence of tight junction formation in an allogeneic graft cell line used for developing an engineered artificial salivary gland. Tissue Eng 8, 871-878
5. Jean-Gilles, R., Soscia, D., Sequeira, S. J., Melfi, M., Gadre, A., Castracane, J., and Larsen, M. (2010) Novel modeling approach to generate nanofibers of defined diameter as scaffolds for salivary gland cells. Journal of Nanotechnology in Engineering and Medicine 1, 031008
6. Guilak, F., Cohen, D. M., Estes, B. T., Gimble, J. M., Liedtke, W., and Chen, C. S. (2009) Control of stem cell fate by physical interactions with the extracellular matrix. Cell Stem Cell 5, 17-26
7. Walles, T., Puschmann, C., Haverich, A., and Mertsching, H. (2003) Acellular scaffold implantation—no alternative to tissue engineering. The International journal of artificial organs 26, 225-234
8. Petersen, T. H., Calle, E. A., Zhao, L., Lee, E. J., Gui, L., Raredon, M. B., Gavrilov, K., Yi, T., Zhuang, Z. W., Breuer, C., Herzog, E., and Niklason, L. E. (2010) Tissue-engineered lungs for in vivo implantation. Science (New York, N.Y. 329, 538-541
9. Oh, S., Brammer, K. S., Li, Y. S., Teng, D., Engler, A. J., Chien, S., and Jin, S. (2009) Stem cell fate dictated solely by altered nanotube dimension. Proceedings of the National Academy of Sciences of the United States of America 106, 2130-2135
10. Biggs, M. J., Richards, R. G., and Dalby, M. J. (2010) Nanotopographical modification: a regulator of cellular function through focal adhesions. Nanomedicine
11. Christopherson, G. T., Song, H., and Mao, H. Q. (2009) The influence of fiber diameter of electrospun substrates on neural stem cell differentiation and proliferation. Biomaterials 30, 556-564
12. Geiger, B., Spatz, J. P., and Bershadsky, A. D. (2009) Environmental sensing through focal adhesions. Nat Rev Mol Cell Biol 10, 21-33
13. Cukierman, E., Pankov, R., Stevens, D. R., and Yamada, K. M. (2001) Taking cell-matrix adhesions to the third dimension. Science (New York, N.Y. 294, 1708-1712
14. Sequeira, S. J., Soscia, D., Oztan, B., Jean-Gilles, R., Gadre, A., Yener, B., Castracane, J., and Larsen, M. (2011) Nanoscale topography of 3D artificial scaffolds regulates salivary gland epithelial cell morphology and focal adhesion complex formation. (in preparation)
15. Krane, C. M., Melvin, J. E., Nguyen, H. V., Richardson, L., Towne, J. E., Doetschman, T., and Menon, A. G. (2001) Salivary acinar cells from aquaporin 5-deficient mice have decreased membrane water permeability and altered cell volume regulation. The Journal of biological chemistry 276, 23413-23420
16. Zheng, C., Hoffman, M. P., McMillan, T., Kleinman, H. K., and O'Connell, B. C. (1998) Growth factor regulation of the amylase promoter in a differentiating salivary acinar cell line. Journal of cellular physiology 177, 628-635
17. Laoide, B. M., Courty, Y., Gastinne, I., Thibaut, C., Kellermann, O., and Rougeon, F. (1996) Immortalised mouse submandibular epithelial cell lines retain polarised structural and functional properties. J Cell Sci 109 (Pt 12), 2789-2800
18. Bockman, C. S., Bruchas, M. R., Zeng, W., O'Connell, K. A., Abel, P. W., Scofield, M. A., and Dowd, F. J. (2004) Submandibular gland acinar cells express multiple alpha1-adrenoceptor subtypes. J Pharmacol Exp Ther 311, 364-372
19. Quissell, D. O., Barzen, K. A., Gruenert, D. C., Redman, R. S., Camden, J. M., and Turner, J. T. (1997) Development and characterization of SV40 immortalized rat submandibular acinar cell lines. In vitro cellular & developmental biology. Animal 33, 164-173
20. Bruchas, M. R., Toews, M. L., Bockman, C. S., and Abel, P. W. (2008) Characterization of the alpha1-adrenoceptor subtype activating extracellular signal-regulated kinase in submandibular gland acinar cells. Eur J Pharmacol 578, 349-358
21. Daley, W. P., Gulfo, K. M., Sequeira, S. J., and Larsen, M. (2009) Identification of a mechanochemical checkpoint and negative feedback loop regulating branching morphogenesis. Dev Biol 336, 169-182
22. Larsen, M., Hoffman, M. P., Sakai, T., Neibaur, J. C., Mitchell, J. M., and Yamada, K. M. (2003) Role of PI 3-kinase and PIP3 in submandibular gland branching morphogenesis. Dev Biol 255, 178-191
23. Larsen, M., Wei, C., and Yamada, K. M. (2006) Cell and fibronectin dynamics during branching morphogenesis. Journal of cell science 119, 3376-3384
24. Chaurey, V., Chiang, P. C., Polanco, C., Su, Y. H., Chou, C. F., and Swami, N. S. (2010) Interplay of electrical forces for alignment of sub-100 nm electrospun nanofibers on insulator gap collectors. Langmuir 26, 19022-19026
25. Lee, J. H., Altemus, B., Xue, Y., Castracane, J., and Gadre, A. (2009) Fabrication and characterization of aligned continuous polymeric electrospun nanofibers. Micro and Nanosystems 1, 116-122
26. Smith, M. J., Smith, D. C., White, K. L., and Bowlin, G. L. (2007) Immune Response Testing of Electrospun Polymers: An Important Consideration in the Evaluation of Biomaterials. Journal of Engineered Fibers and Fabrics 2, 41-47
27. Walker, J. L., Menko, A. S., Khalil, S., Rebustini, I., Hoffman, M. P., Kreidberg, J. A., and Kukuruzinska, M. A. (2008) Diverse roles of E-cadherin in the morphogenesis of the submandibular gland: insights into the formation of acinar and ductal structures. Dev Dyn 237, 3128-3141
28. Larsen, H. S., Aure, M. H., Peters, S. B., Larsen, M., Messelt, E. B., and Galtung, H. K. (2010) Localization of AQP5 during development of the mouse submandibular salivary gland. Journal of Molecular Histology, 1-11
29. Zinzen, K. M., Hand, A. R., Yankova, M., Ball, W. D., and Mirels, L. (2004) Molecular cloning and characterization of the neonatal rat and mouse submandibular gland protein SMGC. Gene 334, 23-33
30. Yixiang, D., Yong, T., Liao, S., Chan, C. K., and Ramakrishna, S. (2008) Degradation of electrospun nanofiber scaffold by short wave length ultraviolet radiation treatment and its potential applications in tissue engineering. Tissue Eng Part A 14, 1321-1329
31. Smith, A. M., Harris, J. J., Shelton, R. M., and Perrie, Y. (2007) 3D culture of bone-derived cells immobilised in alginate following light-triggered gelation. J Control Release
32. Buxboim, A., and Discher, D. E. (2010) Stem cells feel the difference. Nature methods 7, 695-697
33. Buxboim, A., Rajagopal, K., Brown, A. E., and Discher, D. E. (2010) How deeply cells feel: methods for thin gels. J Phys Condens Matter 22
34. Sanz-Herrera, J. A., Moreo, P., Garcia-Aznar, J. M., and Doblare, M. (2009) On the effect of substrate curvature on cell mechanics. Biomaterials 30, 6674-6686
35. Gao, L., McBeath, R., and Chen, C. S. (2010) Stem cell shape regulates a chondrogenic versus myogenic fate through Rac1 and N-cadherin. Stem Cells 28, 564-572
36. Shaw, K. R., Wrobel, C. N., and Brugge, J. S. (2004) Use of three-dimensional basement membrane cultures to model oncogene-induced changes in mammary epithelial morphogenesis. J Mammary Gland Biol Neoplasia 9, 297-310
37. Bissell, M. J., Rizki, A., and Mian, I. S. (2003) Tissue architecture: the ultimate regulator of breast epithelial function. Curr Opin Cell Biol 15, 753-762
38. Tsukita, S., Furuse, M., and Itoh, M. (2001) Multifunctional strands in tight junctions. Nat Rev Mol Cell Biol 2, 285-293
39. Roy, E., Voisin, B., Gravel, J.-F., Peytavi, R., Boudreau, D., and Veres, T. (2009) Microlens array fabrication by enhanced thermal reflow process: Towards efficient collection of fluorescence light from microarrays. Microelectronic Engineering 86, 2255-2261
40. Mosmann, T. (1983) Rapid colorimetric assay for cellular growth and survival: application to proliferation and cytotoxicity assays. Journal of immunological methods 65, 55-63
41. Suzuki, A., and Ohno, S. (2006) The PAR-aPKC system: lessons in polarity. Journal of cell science 119, 979-987
42. Miyoshi, J., and Takai, Y. (2008) Structural and functional associations of apical junctions with cytoskeleton. Biochimica et biophysica acta 1778, 670-691
43. Kawedia, J. D., Nieman, M. L., Boivin, G. P., Melvin, J. E., Kikuchi, K., Hand, A. R., Lorenz, J. N., and Menon, A. G. (2007) Interaction between transcellular and paracellular water transport pathways through Aquaporin 5 and the tight junction complex. Proceedings of the National Academy of Sciences of the United States of America 104, 3621-3626
44. Baker, O. J. (2010) Tight Junctions in Salivary Epithelium. Journal of Biomedicine and Biotechnology 2010
45. Kikuchi, K., Kawedia, J., Menon, A. G., and Hand, A. R. (2010) The structure of tight junctions in mouse submandibular gland. Anat Rec (Hoboken) 293, 141-149
46. Sequeira, S. J., Larsen, M., and DeVine, T. (2010) Extracellular matrix and growth factors in salivary gland development. Frontiers of oral biology 14, 48-77
47. Cantara, S. I., Soscia, D. A., Sequeira, S. J., Jean-Gilles, R. P., Castracane, J., Larsen, M. (2012) Selective functionalization of nanofiber scaffolds to regulate salivary gland epithelial cell proliferation and polarity. Biomaterials 33, 8372-8382

What is claimed is:

1. A cell support comprising a substrate having a concave surface and a nanofiber structure disposed upon said concave surface of said substrate, wherein said nanofiber structure conforms to the concave surface of the substrate.

2. The cell support of claim 1, wherein the nanofiber structure is an electrospun nanofiber scaffold.

3. The cell support of claim 1, wherein said nanofiber structure is removably attached to said substrate.

4. The cell support of claim 1, wherein said concave surface of said substrate has a curvature with an aspect ratio in the range of about 0.5 to about 1.

5. The cell support of claim 1, wherein e of said concave surface has an aspect ratio in the range of about 0.8 to about 1.

6. The cell support of claim 2, wherein a nanofiber of the electrospun nanofiber scaffold has an average diameter in the range of about 50 nm to about 1000 nm.

7. The cell support of claim 2, wherein a nanofiber of the electrospun nanofiber scaffold has an average diameter in the range of about 100 nm to about 500 nm.

8. The cell support of claim 2, wherein a nanofiber of the electrospun nanofiber scaffold has an average diameter in the range of about 150 nm to about 250 nm.

9. The cell support of claim 1, wherein the nanofiber scaffold comprises a natural polymer selected from the group consisting of silk and laminin.

10. The cell support of claim 1, wherein the nanofiber structure comprises a biodegradable polymer selected from the group consisting of poly(∈-caprolactone) (PCL), poly(∈-caprolactone-co-ethyl ethylene phosphate) (PCLEEP), poly(lactic acid) (PLA), poly(lactic-co-glycolic acid) (PLGA), poly(lactic acid-co-∈-caprolactone) (PLACL), and polydioxanone (PDO).

11. The cell support of claim 1, wherein the nanofiber structure comprises PLGA.

12. The cell support of claim 1, wherein the nanofiber structure comprises a non-biodegradable polymer selected from the group consisting of: poly acrylamide (PAAm), poly acrylic acid (PAA), poly acrylonitrile (PAN), poly amide (Nylon) (PA, PA-4,6, PA-6,6), poly aniline (PANI), poly benzimidazole (PBI), poly bis(2,2,2-trifluoroethoxy) phosphazene, poly butadiene (PB), poly carbonate (PC), poly ether amide (PEA), poly ether imide (PEI), poly ether sulfone (PES), poly ethylene (PE), poly ethylene-co-vinyl acetate (PEVA), poly ethylene glycol (PEG), poly ethylene oxide (PEO), poly ethylene terephthalate (PET), poly ferrocenyldimethylsilane (PFDMS), poly 2-hydroxyethyl methacrylate (HEMA), poly 4-methyl-1-pentene (TpX), poly methyl methacrylate (pMMA), poly p-phenylene terephthalamide (PPTA), poly propylene (PP), poly pyrrole (PPY), poly styrene (PS), polybisphenol-A sulfone (PSF), poly sulfonated styrene (PSS), Styrene-butadiene-styrene triblock copolymer (SBS), poly urethane (PU), poly tetrafluoro ethylene (PTFE), poly vinyl alcohol (PVA), poly vinyl carbazole, poly vinyl chloride (PVC), poly vinyl phenol (PVP), poly vinyl pyrrolidone (PVP), and poly vinylidene difluoride (PVDF).

13. The cell support of claim 1, wherein the nanofiber structure further comprises one or more biomolecules covalently linked to said nanofiber.

14. The cell support of claim 1, wherein said biomolecule is selected from the group consisting of fibronectin, collagen, elastin, laminin, and perlecan.

15. The cell support of claim 1, wherein said nanofiber structure has a pore size between about 1.00 μm and about 5.00 μm.

16. The cell support of claim 1, wherein said nanofiber structure has a pore size between about 1.50 μm and about 4.00 μm.

17. The cell support of claim 1, wherein said nanofiber structure has a pore size between about 1.80 μm and about 3.40 μm.

18. The cell support of claim 1, wherein said nanofiber structure has a thickness between about 0.5 μm and 2.0 μm.

19. The cell support of claim 1, wherein said nanofiber structure has a thickness between about 0.7 μm and 1.65 μm.

20. A method for promoting differentiation and morphogenesis of secretory epithelial cells ex vivo, the method comprising:
a. seeding secretory epithelial cells on the concave surface of a cell support comprising a substrate with a concave surface and a nanofiber structure disposed upon said concave surface of said substrate, wherein said nanofiber structure conforms to the curvature of the concave surface;
b. incubating said cell support and cells under conditions sufficient for proliferation, differentiation and morphogenesis of said cells into acini.

21. A method for screening a compound that modulates development of secretory epithelial cells, the method comprising:
a. seeding secretory epithelial cells on a cell support comprising a substrate with a concave surface and a nanofiber structure disposed upon said concave surface of said substrate, wherein said nanofiber structure conforms to the concave surface;
b. incubating said cells and said support under conditions sufficient for growth, differentiation and morphogenesis of said cells in the absence and presence of the compound to be tested;
c. monitoring development of said cells grown in the presence of said compound and monitoring development of the cells grown in the absence of said compound; and
d. comparing the development of the cells grown in the presence of said compound and cells grown in the absence of said compound.

22. The method of claim 21, wherein said monitoring step comprises
a. evaluating cell morphology;
b. evaluating expression and/or location of one or more differentiation markers in said cells;
c. detection of organ product; or
d. any combination of a., b. and c.

23. The method of claim 21, wherein the cells are salivary gland cells.

24. The method of claim 21, wherein the cells are pancreatic cells.

25. The method of claim 23, wherein the differentiation marker is selected from aquaporin-5 (Aqp-5), α-amylase, occludin, E-cadherin and zonula occludens-1 (ZO-1).

26. The method of claim 23, wherein the organ product is saliva.

27. The method of claim 29, wherein the organ product is salivary amylase-binding protein A (SABPA).

28. A device comprising a substrate having a plurality of concave surfaces and a nanofiber structure disposed upon each of said concave surfaces of said plurality of concave surfaces, wherein each of said nanofiber structures conforms to the concave surfaces of the substrate.

29. The device of claim 28, wherein the nanofiber structure is an electrospun nanofiber scaffold.

30. The device of claim 28, wherein each of said concave surfaces has a curvature with an aspect ratio in the range of about 0.5 to about 1.

31. The device of claim 28, wherein each of said concave surfaces has a curvature with an aspect ratio in the range of about 0.8 to about 1.

32. The device of claim 28, wherein a nanofiber of the electrospun nanofiber scaffold has an average diameter in the range of about 50 nm to about 1000 nm.

33. The device of claim 28, wherein a nanofiber of the electrospun nanofiber scaffold has an average diameter in the range of about 100 nm to about 500 nm.

34. The device of claim 28, wherein a nanofiber of the electrospun nanofiber scaffold has an average diameter in the range of about 150 nm to about 250 nm.

35. The device of claim 28, wherein the nanofiber scaffold comprises a natural polymer selected from the group consisting of silk and laminin.

36. The device of claim 28 wherein the nanofiber structure comprises a biodegradable polymer selected from the group consisting of poly(∈-caprolactone) (PCL), poly(∈-caprolactone-co-ethyl ethylene phosphate) (PCLEEP), poly(lactic acid) (PLA), poly(lactic-co-glycolic acid) (PLGA), poly(lactic acid-co-∈-caprolactone) (PLACL), and polydioxanone (PDO).

37. The device of claim 28, wherein the nanofiber structure comprises PLGA.

38. The device of claim 28, wherein the nanofiber structure comprises a non-biodegradable polymer selected from the group consisting of: poly acrylamide (PAAm), poly acrylic acid (PAA), poly acrylonitrile (PAN), poly amide (Nylon) (PA, PA-4,6, PA-6,6), poly aniline (PANI), poly benzimidazole (PBI), poly bis(2,2,2-trifluoroethoxy) phosphazene, poly butadiene (PB), poly carbonate (PC), poly ether amide (PEA), poly ether imide (PEI), poly ether sulfone (PES), poly ethylene (PE), poly ethylene-co-vinyl acetate (PEVA), poly ethylene glycol (PEG), poly ethylene oxide (PEO), poly ethylene terephthalate (PET), poly ferrocenyldimethylsilane (PFDMS), poly 2-hydroxyethyl methacrylate (HEMA), poly 4-methyl-1-pentene (TpX), poly methyl methacrylate (pMMA), poly p-phenylene terephthalamide (PPTA), poly propylene (PP), poly pyrrole (PPY), poly styrene (PS), polybisphenol-A sulfone (PSF), poly sulfonated styrene (PSS), Styrene-butadiene-styrene triblock copolymer (SBS), poly urethane (PU), poly tetrafluoro ethylene (PTFE), poly vinyl alcohol (PVA), poly vinyl carbazole, poly vinyl chloride (PVC), poly vinyl phenol (PVP), poly vinyl pyrrolidone (PVP), and poly vinylidene difluoride (PVDF).

39. The device of claim 28, wherein the nanofiber structure further comprises one or more biomolecules covalently linked to said nanofiber.

40. The device of claim 28, wherein said biomolecule is selected from the group consisting of fibronectin, collagen, elastin, laminin, chitosan and perlacan.

41. The device of claim 28, wherein said nanofiber structure has a pore size between about 1.00 μm and about 5.00 μm.

42. The device of claim 28, wherein said nanofiber structure has a pore size between about 1.50 μm and about 4.00 μm.

43. The device of claim 28, wherein said nanofiber structure has a pore size between about 1.80 μm and about 3.40 μm.

44. The device of claim 28, wherein said nanofiber structure has a thickness between about 0.5 μm and 2.0 μm.

45. The device of claim 28, wherein said nanofiber structure has a thickness between about 0.7 μm and 1.65 μm.
